(19) United States
(12) Patent Application Publication
(10) Pub. No.: US 2011/0281814 A1
(43) Pub. Date: Nov. 17, 2011

(54) METHODS AND COMPOSITIONS FOR TREATING BREAST CANCER

(75) Inventors: Ajeeta Dash, Southborough, MA (US); Ross Tubo, Quincy, MA (US)

(73) Assignee: Genzyme Corporation

(21) Appl. No.: 13/146,287

(22) PCT Filed: Jan. 28, 2010
(86) PCT No.: PCT/US2010/022425

§ 371 Date: Jul. 26, 2011

Related U.S. Application Data
(60) Provisional application No. 61/148,898, filed on Jan. 30, 2009.

(51) Int. Cl. A61K 31/704; A61K 31/4427; A61P 35/00; A61K 31/395

(52) U.S. Cl. 514/34; 514/183; 514/340

(57) ABSTRACT

The invention relates to methods and compositions for treating a subject afflicted with breast cancer using a CX-CR4 antagonist and optionally in combination with a chemotherapeutic agent.

### TECHNICAL FIELD

This invention is in the field of treating breast cancer. In particular, the invention concerns methods and compositions for treating a subject afflicted with breast cancer using a CXCR4 antagonist and optionally in combination with a chemotherapeutic agent.

### BACKGROUND ART

Although its mortality rate has been declining steadily in the recent years due to early detection and to improvements in treatment, breast cancer remains the fifth leading cause of cancer death, accounting for 548,000 deaths worldwide in 2007. (See World Health Organization, Fact Sheet No. 297, July 2008). A common approach to treating breast cancer is surgery combined with radiation therapy and/or systemic therapy, which usually involves chemotherapy, hormone therapy and/or biologic therapy (e.g., monoclonal antibodies).

The benefit of chemotherapy is dependent on multiple factors including the size of the cancer, the number of lymph nodes involved, the presence of estrogen and progesterone receptors, and the amount of the HER2/neu tyrosine kinase receptor produced by the cancer cells. The most common chemotherapeutic agents recommended to be used in combination in early breast cancer are cyclophosphamide, methotrexate, fluorouracil, doxorubicin, epirubicin, paclitaxel, and docetaxel. Depending on the combination of drugs that is used, chemotherapy is usually administered for three to six months. These and other chemotherapeutic agents may also be used to treat advanced and metastatic breast cancers.

It is believed that one of the major shortcomings of the conventional chemotherapeutic regimens is the diminished ability of chemotherapy to completely eliminate the malignant cells and/or their precursors. The present invention addresses this problem by combining chemotherapy with the administration of CXCR4 antagonists.

The compounds useful in the method of the invention are antagonists of the CXCR4 receptor that prevent its interaction with the cytokine stromal cell derived factor-1 (SDF-1), which is now designated as CXCL12. Many such agents and uses of such agents are known in the art. One notable agent is 1,1′-[1,4-phenylene-bis-(methylene)-bis-1,4,8,11-tetraazacyclotetradecane (also known by its codename, AMD3100), which is the active ingredient of MOZOBIL® (plerixafor), which is approved by the FDA for use in combination with granulocyte-colony stimulating factor (G-CSF) to mobilize hematopoietic stem cells to the peripheral blood for collection and subsequent autologous transplantation in patients with non-Hodgkin's lymphoma (NHL) and multiple myeloma (MM). This CXCR4 antagonist and other CXCR4 antagonists are disclosed, for example, in U.S. Pat. Nos. 5,021,409; 6,001,826; 5,583,131; 5,698,546; 5,817,807; 6,506,770; 6,756,391; 7,160,872; 6,872,714; 7,414,065; 6,667,320 and 7,022,717; in U.S. Patent Application Pub. Nos. 2007/0043012 and 2007/0060591; and in PCT Pub. Nos. WO 92/016494; WO 93/012096; WO 95/018808; WO 00/002870 and WO 01/044229, all of which are incorporated herein by reference.

The chemokine receptor CXCR4 and its natural ligand SDF-1α/CXCL12 play pleiotropic roles in angiogenesis, host immune response, homing and tumor metastasis. As early as 2001, it was shown that CXCR4 was highly expressed in malignant breast cancer cells but not in normal breast tissue, and the ligand SDF-1α/CXCL12 was highly expressed in bone marrow, lung and lymph nodes, where breast cancer cells metastasize preferentially (Müller A. et al., Nature (2001) 410:50-56). It was further demonstrated that neutralizing the interactions of CRCX4 and SDF-1/CXCL12 in vivo with a specific monoclonal antibody against CXCR4 effectively inhibited metastasis of breast cancer cells to the regional lymph nodes and lung (Id.). Tamamura et al. showed that peptidic antagonists of CXCR4 effectively inhibited SDF-1α-induced migration of human breast cancer cells (MDA-MB-231) in vitro and that slow release administration of these compounds by subcutaneous injection reduced pulmonary metastasis of breast cancer cells in SCID mice (Tamamura, H. et al., FEBS Lett. (2003) 550:79-83).

Numerous gene expression studies showed significant correlations between the expression and localization patterns of CXCR4 and the degree of lymph node metastasis (Kato, M. et al., Breast Cancer Res. (2003) 5:R144-R150; Cabioglu, N. et al., Clin. Cancer Res. (2005) 11:5686-5693; Kang, H. et al., Breast (2005) 14:360-367; Blot, E. et al., Breast (2008) 14:268-274; Woo, S. U. et al., Ann. Surg. Oncol. (2008) 15:281-285), HER2/neu-mediated metastasis (Li, Y. M. et al., Cancer Cell (2004) 6:459-469), tumor grade (Salvucci, O. et al., Breast Cancer Res. Treat. (2006) 97:275-83), likelihood of recurrence (Holm, N. T. et al., J. Surg. Res. (2007) 141:53-59) and poor clinical outcome (Cabioglu, N. et al., Ann. Oncol. (2007) 18:1021-1029; Chu, Q. D. et al., J. Surg. Res., doi:10.1016/j.jss.2008.09.020, prepublished online Oct. 16, 2008). It was also found that the expression of CXCR4 is negatively regulated by tumor suppressor p53 in breast cancer cells (Mehta, S. A. et al., Oncogene (2007) 26:3329-3337) and positively regulated by HER2/neu (see Li et al. supra) and nitric oxide (Yasuoka, H. et al., BMC Cancer (2008) 8:340).

Similarly, it has been established that expression level of SDF-1α/CXCL12 is significantly higher in human breast cancer epithelial cancer cells compared with normal epithelial cells (Lee, B. C. et al., Mol. Cancer. Res. (2004) 2:327-338), and that expression levels of SDF-1α/CXCL12 show a significant correlation with lymph node involvement and long-term survival in breast cancer patients (Kang, H. et al., Breast Cancer Res. (2005) 7:R402-R410). Interestingly, it was found that SDF-1α/CXCL12 transactivated HER2/neu in the breast cancer cell lines MDA-MB-361 and SKBR3, which express both CXCR4 and HER2/neu, through a novel pathway involving Src kinase activation (Cabioglu, N. et al., Cancer Res. (2005) 65:6493-6497).

A number of different studies have shown that inhibition of CRCX4 function in breast cancer cells leads to decreased cell migration in vitro and lower cancer invasiveness in vivo. For example, it was demonstrated that inducible knockdown of endogenous CXCR4 gene expression in metastatic breast cancer cells by RNA interference (siRNA) prevented their migration toward SDF-1α in vitro (Chen, Y. et al., Cancer Res. (2003) 63:4801-4804) and inhibited breast cancer growth and metastasis in mouse tumor models (Smith, M. C. P. et al., Cancer Res. (2004) 64:8604-8612; Lapteva, N. et al., Cancer Gene Ther. (2005) 12:84-89; Liang, Z. et al., Cancer Res. (2005) 65:967-971; Liang, Z. et al., Biochem. Biophys. Res. Commun. (2007) 363:542-546). Additionally, administration of selective peptidomimetic CXCR4 receptor antagonists such as TN14003 and CTCE-9908 has been shown to reduce metastasis and reduce primary tumor growth in mouse models of breast cancer (see Tamamura et al. supra; Liang, Z. et al., Cancer Res. (2004) 64:4302-4308; Huang, E. et al., J. Surg. Res., doi:10.1016/j.jss.2008.06.044, prepublished online Aug. 11, 2008). AMD3100 is a cyclic polyamine chemokine receptor antagonist specific for CXCR4 (see Hatse, S. et al., FEBS Lett. (2002) 527:255-262; De Clercq, E. Nat. Rev. Drug Discov. (2003) 2:581-587). In the in vitro context, AMD3100 was shown to inhibit the SDF-1α/CXCL12-mediated transactivation of HER2/neu in the MDA-MB-361 breast cancer cells (see Cabioglu, N. et al., Cancer Res. (2005) 65:6493-6497). In vivo, AMD3100 significantly delayed the growth onset of murine 4T1 cells in the lung, although it did not appear to improve overall survival in mice with experimental lung metastases (see Smith et al. supra).

We have previously found, and disclosed in PCT Pub. No. WO 00/045814, that the certain CXCR4 antagonists, such as AMD3100, have the effect of increasing the white blood cell count. We have also found, and have disclosed in PCT Pub. No. WO 03/011277, that these antagonists have the effect of mobilizing progenitor cells and/or stem cells from the bone marrow to the circulating blood. Certain uses of CXCR4 antagonists are disclosed in U.S. Patent Application Pub. Nos. 2007/0043012 and 2007/0060591; and PCT Pub. No. WO 08/019,371, all of which are incorporated herein by reference. U.S. Patent Application Pub. No. 2007/0043012, commonly assigned to the current applicant, discloses the use of CXCR4 antagonists to potentiate the effects of standard chemotherapeutic agents through the release and/or rapid movement of pre-leukemic cells and leukemic cells from the microenvironment of the bone marrow and into the circulating blood prior to, or during, or after treatment by chemotherapy. U.S. Patent Application Pub. No. 2007/0043012 does not specifically mention the use of CXCR4 antagonists to potentiate the effects of chemotherapeutic agents in the context of breast cancer treatment, and does not specifically mention certain of the immunotherapeutic agents disclosed in the present invention.

There is currently a need for alternative or improved treatments of breast cancer. The current invention addresses such need by use of antagonists of the CXCR4 receptor alone or in combination with chemotherapeutic agents, and it surprisingly has been found as demonstrated by the data presented herein that the combination of CXCR4 antagonists with a chemotherapeutic agent is of potential clinical significance. Moreover, we surprisingly have found that the dose of the CXCR4 antagonists is related to these findings insofar as a lower dose of the CXCR4 antagonist is associated with a greater potential therapeutic benefit.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents, and not intended to be bound by any theory or hypothesis described in these documents. Further, all documents referred to throughout this application are incorporated in their entirety by reference herein.

### DISCLOSURE OF THE INVENTION

In one aspect, the invention is directed to a method for treating a subject afflicted with breast cancer, which comprises administering a therapeutically effective amount of a CXCR4 antagonist as defined below in combination with a chemotherapeutic agent. The CXCR4 antagonist may be administered prior to, during, and/or after the chemotherapeutic regimen is administered.

In certain embodiments, the CXCR4 antagonist comprises a compound of the formula:

Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula

—N(R)—(CR₂)ₙ—X
wherein each R is independently H or straight, branched or cyclic alkyl (1-6C), n is 1 or 2, and X is an aromatic ring, including heteroaromatic rings, or is a mercaptan,
or Z′ may be of the formula

—Ar(Y)ⱼ
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

In certain embodiments, the CXCR4 antagonist comprises a compound of the formula:

Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula

—N(R)—(CR₂)ₙ—X
wherein each R is independently H or straight, branched or cyclic alkyl (1-6C), n is 1 or 2, and X is an aromatic ring, including heteroaromatic rings, or is a mercaptan; and
“linker” represents a bond, alkylene (1-6C) or may comprise aryl, fused aryl, oxygen atoms contained in an alkylene chain, or may contain keto groups or nitrogen or sulfur atoms.

In another aspect, the invention is directed to a method for treating a subject afflicted with breast cancer, which comprises administering a therapeutically effective amount of a CXCR4 antagonist.

In another aspect, the invention is directed to a pharmaceutical or veterinary composition comprising a CXCR4 antagonist in unit dosage form for use in the methods of the invention. In certain embodiments, the composition comprises a CXCR4 antagonist, a chemotherapeutic agent and a suitable pharmaceutically or veterinary acceptable excipient. In certain embodiments, the CXCR4 antagonist comprises a compound of formula (1). In certain other embodiments, the CXCR4 antagonist is one disclosed herein.

Small molecule CXCR4 antagonists useful in the present invention are disclosed in U.S. Pat. Nos. 5,021,409; 6,001,826; 5,583,131; 5,698,546; 5,817,807; 6,506,770; 6,756,391; 7,160,872; 6,872,714; 7,414,065; 6,667,320 and 7,022,717; in U.S. Patent Application Pub. Nos. 2007/0043012 and 2007/0060591; and in PCT Pub. Nos. WO 92/016494; WO 93/012096; WO 95/018808; WO 00/002870 and WO 01/044229, all incorporated herein by reference.

In other embodiments, peptide-based antagonists may be used. These are described in PCT Pub. Nos. WO 01/85196; WO 00/09152 and WO 99/47158. The use of antibodies as antagonists of CXCR4 interacting with its ligand are disclosed in WO 99/50461. Other peptide-based compounds include T22 (Murakami, T. et al., J. Exp. Med. (1997) 186:1389-1393); T134 (Arakaki, R. et al., J. Virol. (1999) 73:1719-1723; T140 (Tamamura, H. et al., Biochem. Biophys. Res. Comm. (1998) 253:877-882) and its analogs TC14012 and TN14003 (Tamamura, H. et al., Bioorg. Med. Chem. Lett. (2001) 11:1897-1902; Mori, T. et al., Mol. Cancer. Ther. (2004) 3:29-37; Burger, M. et al., Blood (2005) 106:1824-1830); ALX40-4C (Doranz, B. J. et al., J. Exp. Med. (1997) 186:1395-1400; Donzella, G. A., Nat. Med. (1998) 4:72-77; Doranz, B. J. et al., AIDS Res. Hum. Retrovir. (2001) 17:475-486); RCP168 (Zeng, Z. et al., Mol. Cancer. Ther. (2006) 5:3113-3121); CTCE-0021 (Pelus, L. M. et al., Exp. Hematol. (2005) 33:295-307); CTCE-0214 (Thong, R. et al., Exp. Hematol. (2004) 32:470-475); CTCE-9908 (Kim, S. Y. et al., AACR Meeting Abstracts (2005) Abstract 256); KRH-1120 (Yamamoto, N. et al., J. AIDS Res. (2000) 2:453-460); KRH-1636 (Ichiyama, K. et al., Proc. Natl. Acad. Sci. USA (2003) 100:4185-4190); KRH-2731 (Murakami T. et al., Abstracts of the 11th Conference on Retroviruses and Opportunistic Infections (2004) Abstract 541); and CXCR4 antagonists described in PCT Pub. Nos. WO 99/47158; WO 99/50461; WO 00/09152; WO 01/85196; WO 01/94420; and WO 03/090512. As to the methods for preparation of these substances, they can be found, for example, in J. Exp. Med. (1997) 186:1189-1191 with any conventional modifications. All of these publications are incorporated herein by reference in their entireties.

### BRIEF DESCRIPTION OF THE DRAWINGS

FIG. 1 illustrates in vivo therapeutic efficacy of AMD3465 in severe combined immunodeficiency (SCID) mice injected with MDA-MB-231 human breast cancer cells and human mesenchymal stem (MSC) cells. AMD3465 significantly decreased the rate of tumor growth at 5 mg/kg but not at 10 mg/kg body weight.

FIGS. 2A and 2B compare in vivo therapeutic efficacy of AMD3465 in SCID mice injected with MDA-MB-231 cells in the presence or absence of stromal MSC cells, respectively. FIG. 2A shows that AMD3465 significantly decreased the rate of tumor growth at 5 mg/kg but not at 10 mg/kg in mice injected with MDA-MB-231 and MSC cells. FIG. 2B shows that AMD3465 had no statistically significant effect at either concentration in mice injected with MDA-MB-231 cells only.

FIGS. 3A and 3B illustrate in vivo therapeutic efficacy of AMD3465 in established MDA-MB-231 tumors. FIG. 3A shows that AMD3465 significantly decreased the rate of tumor growth at 2.5 mg/kg but not at 5 or 10 mg/kg in mice injected with MDA-MB-231 cells. FIG. 3B shows that AMD3465 also increased the mean survival at 2.5 mg/kg but not at 5 or 10 mg/kg in mice injected with MDA-MB-231 cells.

FIGS. 4A and 4B illustrate in vivo therapeutic efficacy of AMD3100 in established 4T1 tumors. FIG. 4A shows that AMD3100 significantly decreased the rate of tumor growth at 1.25 mg/kg but not at 2.5 mg/kg in mice injected with 4T1 cells. FIG. 4B shows that AMD3100 also increased the mean survival at 1.25 mg/kg but not at 2.5 mg/kg in mice injected with 4T1 cells.

FIG. 5 illustrates in vivo therapeutic efficacy of AMD3100 in combination with doxorubicin. The combination of 2.5 mg/kg AMD3100 and 2 mg/kg doxorubicin delayed the onset of active tumor growth and ultimately resulted in smaller tumor volumes compared with 2 mg/kg doxorubicin alone.

FIGS. 6A and 6B represent another illustration of in vivo therapeutic efficacy of AMD3100 in combination with doxorubicin. FIG. 6A shows that the combination of 2.5 mg/kg AMD3100 and 1 mg/kg doxorubicin significantly delayed the onset of active tumor growth, reduced growth rates and ultimately resulted in smaller tumor volumes compared with 1 mg/kg doxorubicin alone. FIG. 6B shows that the combination of 2.5 mg/kg AMD3100 and 1 mg/kg doxorubicin also significantly increased the mean survival compared to doxorubicin alone.

### MODES OF CARRYING OUT THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

The discussion of the general methods given herein is intended for illustrative purposes only. Other alternative methods and embodiments will be apparent to those of skill in the art upon review of this disclosure.

As used herein, “a” or “an” means “at least one” or “one or more.”

A group of items linked with the conjunction “or” should not be read as requiring mutual exclusivity among that group, but rather should also be read as “and/or” unless expressly stated otherwise.

As used herein, the terms “treatment” or “treating” refers to any manner in which the symptoms of a condition, disorder or disease are ameliorated or otherwise beneficially altered. In the context of treating breast cancer, the breast cancer can be onset, relapsed or refractory. In addition, treatment includes a reduction of tumor size, irrespective of stage, as well as a prevention of metastasis. Full eradication of the condition, disorder or disease is not required. Amelioration of symptoms of a particular disorder refers to any lessening of symptoms, whether permanent or temporary, that can be attributed to or associated with administration of a therapeutic composition of the present invention or the corresponding methods and combination therapies. Treatment also encompasses pharmaceutical use of the compositions in accordance with the methods disclosed herein.

As used herein, the term “subject” is not limited to a specific species or sample type. For example, the term “subject” may refer to a patient, and frequently a human patient. However, this term is not limited to humans and thus encompasses a variety of mammalian species.

The term “afflicted” as it relates to a disease or disorder refers to a subject having or directly affected by the designated disease or disorder.

As used herein, the term “breast cancer” refers to a malignant tumor that has developed from cells in the breast irrespective of whether such cancer is onset, relapsed or refractory. Breast cancer usually originates in the cells of the lobules, which are the milk-producing glands (lobular carcinoma), or the ducts, the passages that drain milk from the lobules to the nipple (ductal carcinoma). Less commonly, breast cancer can originate in the stromal tissues, which include the fatty and fibrous connective tissues of the breast. Less common types of breast cancer include inflammatory breast cancer, medullary carcinoma, mucinous (colloid) carcinoma, Paget's disease, tubular carcinoma, phylloides tumor, metaplastic carcinoma, sarcoma, micropapillary carcinoma, and adenoid cystic carcinoma.

As used herein, the term “in situ breast cancer” refers to a type of breast cancer wherein the cancer cells remain within their site of origin and do not attack breast tissue around the duct or lobule. The most common types of in situ breast cancer are ductal carcinoma in situ (DCIS) and lobular carcinoma in situ (LCIS).

As used herein, the term “invasive breast cancer” refers to a type of breast cancer that dissociates from their site of origin and invades the surrounding tissues. As with in situ breast cancer, the most common types of invasive breast cancer are invasive ductal carcinoma (IDC) and invasive lobular carcinoma (ILC).

As used herein, the term “metastatic breast cancer” refers to a type of breast cancer that spreads to other organs of the body. The term “node-positive” refers to a type of breast cancer wherein the cancer cells are detectable in the lymph nodes. The term “node-negative” refers to a type of breast cancer wherein the cancer cells are not detectable in the lymph nodes. Breast cancers are further classified as either “HER2-positive” or “HER2-negative” based on the expression levels of the HER2/neu tyrosine kinase receptor.

Breast cancers are staged based on the size and localization of the tumor. “Stage I and II breast cancers” refer to relatively small (less than 2 cm in size for Stage I and less than 5 cm for Stage II), localized, node-negative tumors. “Stage III breast cancer” refers to locally advanced, invasive, node-positive tumors that are greater than 5 cm in size. “Stage IV breast cancer” refers to tumors that have metastasized to areas outside the breast, including the brain, bones, skin, or other organs.

As used herein, the terms “administration” or “administering” refers to any suitable method of providing a composition of the present invention to a subject. It is not intended that the present invention be limited to any particular mode of administration. In certain embodiments, the compounds and pharmaceutical compositions of the present invention are administered by a parenteral route, e.g., via intramuscular, intraperitoneal, intravenous, intracisternal or subcutaneous injection or infusion. The pharmaceutical compositions may be formulated in suitable dosage unit formulations appropriate for each route of administration.

As used herein, the term “effective amount” or “therapeutically effective amount” of a compound refers to a nontoxic but sufficient amount of the compound to provide the desired therapeutic or prophylactic effect to most patients or individuals. In the context of treating breast cancer, a nontoxic amount does not necessarily mean that a toxic agent is not used, but rather means the administration of a tolerable and sufficient amount to provide the desired therapeutic or prophylactic effect to a patient or individual. The effective amount of a pharmacologically active compound may vary depending on the route of administration, as well as the age, weight, and sex of the individual to which the drug or pharmacologically active agent is administered. Those of skill in the art given the benefit of the present disclosure can easily determine appropriate effective amounts by taking into account metabolism, bioavailability, and other factors that affect plasma levels of a compound following administration within the unit dose ranges disclosed further herein for different routes of administration.

As used herein, the term “chemotherapy” generally refers to the use of drugs to treat cancer. As used herein, the term “chemotherapeutic agent” generally refers a compound or pharmaceutical composition that is administered in the treatment of cancer. As used herein, the term “chemotherapy” encompasses both neoadjuvant and adjuvant chemotherapy. The term “neoadjuvant chemotherapy” refers to chemotherapy administered before surgery. The goal of neoadjuvant chemotherapy is to sufficiently reduce the size of the tumor to facilitate its surgical removal or to allow for less extensive surgery. The term “adjuvant chemotherapy” refers to chemotherapy administered after surgery. The purpose of adjuvant chemotherapy is to reduce the risk of relapse and increase the cure rate of the patient.

Chemotherapeutic agents are typically categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into one or more of the following categories: alkyl sulfonates, alkylating agents, antimetabolites, antitumor antibiotics, biological response modifiers, corticosteroid hormones, epipodophyllotoxins, ethylamines, folic acid analogs, hormone agents and antagonists, methylmelamines, mitotic inhibitors, natural products, nitrogen mustards, nitrosoureas, purine analogs, pyrimidine analogs, taxoids, triazines, vinca alkaloids, and any analog or derivative variant thereof.

Chemotherapeutic agents include, but are not limited to: 5-fluorouracil, actinomycin D, adrenocorticoids, adrenalcortical suppressant, amsacrine, aminoglutethimide, anthracenediones, bleomycin, busulfan, camptothecin, carboplatin, caproate, chlorambucil, cisplatin (CDDP), carboplatin, carmustine, clofarabine, cyclophosphamide, cytarabine, dacarbazine, dactinomycin, daunorubicin, dexamethasone, diethylstilbestrol, docetaxel, doxorubicin, epirubicin, epothilones, estrogen receptor binding agents, ethinyl estradiol, etoposide (VP16), farnesyl-protein transferase inhibitors, floxuridine, fludarabine, fluoxymesterone, flutamide, geldanamycin, gemcitabine, hexamethylmelamine, hydroxyprogesterone, hydroxyurea, idarubicin, ifosfamide, irinotecan, L-asparaginase, leuprolide, lomustine, mechlorethamine, medroxyprogesterone acetate, megestrol acetate, melphalan, mercaptopurine, methotrexate, methyl hydrazine derivatives, mithramycin, mitomycin, mitotane, mitoxantrone, navelbine, nitrosurea, paclitaxel, pentostatin, picoplatin, platinum coordination complexes, plicamycin, prednisone, procarbazine, raloxifene, semustine, streptozocin, substituted urea, tamoxifen, taxol (paclitaxel), taxotere (docetaxel), teniposide, testosterone propionate, thioguanine, thiotepa, temazolomide, transplatinum, tretinoin, topotecan, vinblastine, vincristine, vinorelbine, or any analog or derivative thereof.

As noted above, in one aspect, the present invention is concerned with the use of a therapeutically effective amount of a CXCR4 antagonist in combination with a chemotherapeutic agent to treat a subject afflicted with a hematological malignancy.

Since typical chemotherapy approaches ultimately elicit their effects via apoptosis, alterations at the level of apoptosis control provide an effective mechanism by which drug resistance may occur. Drug resistance may emerge if cancer cells adjust the expression levels of certain proteins that regulate the propagation of signals arising from cellular insults, such as chemotherapy, to protect against apoptosis (e.g., p53, Bcl-2 family, IAP family, etc.). While not wishing to be bound by any particular theory or mechanism of action, it is believed that in certain embodiments CXCR4 antagonists may chemosensitize breast cancer cells, i.e., increase the cells' susceptibility to chemotherapeutic agents, by disrupting their apoptosis regulation.

In certain embodiments, the CXCR4 antagonist comprises a compound of the formula:

Z-linker-Z′ (1)
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula

—N(R)—(CR₂)ₙ—X
wherein each R is independently H or straight, branched or cyclic alkyl (1-6C),
n is 1 or 2, and
X is an aromatic ring, including heteroaromatic rings, or is a mercaptan;
or Z′ may be of the formula

—Ar(Y)ⱼ
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

Specific forms of the compounds of formula (1) are discussed below.

In compounds of formula (1), certain embodiments of Z and Z′ are cyclic polyamine moieties having from 9-24C that include 3-5 nitrogen atoms, for example, 1,5,9,13-tetraazacyclohexadecane; 1,5,8,11,14-pentaazacyclohexadecane; 1,4,8,11-tetraazacylotetradecane; 1,4,7-triazacyclotetradecane; 1,5,9-triazacyclododecane; 1,4,7,10-tetraazacyclododecane; and the like, including such cyclic polyamines which are fused to an additional aromatic or heteroaromatic rings and/or containing a heteroatom other than nitrogen incorporated in the ring. Embodiments of Z and Z′ wherein the cyclic polyamine contains a fused additional cyclic system or one or more additional heteroatoms include, for example, 3,7,11,17-tetraazabicyclo(13.3.1)heptadeca-1(17),13,15-triene; 4,7,10,17-tetraazabicyclo[13.3.1]heptadeca-1(17),13,15-triene; 4,7,10-triazabicyclo[13.3.1]heptadeca-1(17),13,15-triene; and 4,10-diazabicyclo[13.31.1]heptadeca-1(17),13,15-triene. These and other related embodiments are described in U.S. Pat. No. 5,698,546 and PCT Pub No. WO 01/44229, incorporated herein by reference.

Embodiments of the linker moiety include those wherein the linker is a bond, or wherein the linker includes an aromatic moiety bracketed by two alkylene, preferably methylene moieties. Linking groups include the methylene bracketed forms of 1,3-phenylene, 2,6-pyridine, 3,5-pyridine, 2,5-thiophene, 4,4′-(2,2′-bipyrimidine); 2,9-(1,10-phenanthroline) and the like. A particularly preferred linker is 1,4-phenylene-bis-(methylene).

In certain embodiments, the compounds include those of formula (1) wherein Z and Z′ are both cyclic polyamines. In certain other embodiments, Z and Z′ are identical. In further embodiments, Z is a cyclic polyamine that contains 10-24 members and contains 4 nitrogen atoms. In some specific embodiments, Z and Z′ are both 1,4,8,11-tetraazocyclotetradecane.

Certain embodiments of the compound of the formula (1) include 2,2′-bicyclam and 6,6′-bicyclam; the embodiments set forth in U.S. Pat. Nos. 5,021,409, and 6,001,826, and in particular 1,1′-[1,4-phenylene-bis(methylene)]-bis-1,4,8,11-tetraazacyclotetradecane, set forth in U.S. Pat. No. 5,583,131, and sometimes designated herein as AMD3100.

When Z′ is other than a cyclic polyamine as defined in Z, certain embodiments are set forth in U.S. Pat. Nos. 5,817,807; 6,506,770; 6,756,391; 7,160,872; 6,872,714; 7,414,065; 6,667,320 and 7,022,717, incorporated herein by reference. In certain other embodiments, Z is 1,4,8,11-tetraazacyclotetradecane, the linker is 1,3- or 1,4-phenylene-bis(alkylene) in particular 1,4-phenylene-bis(methylene) and Z is —NR(CR₂)ₙ—X, where X is pyridine, and in particular wherein Z is NHCH₂CH₂-pyridine. In further embodiments, the compound is N-[1,4,8,11-tetraazacyclotetradecanyl-(1,4-phenylene-bis-(methylene)]-2-aminoalkylpyridine, sometimes designated herein as AMD3465.

In certain embodiments, the CXCR4 antagonist includes, but is not limited to, linear peptides, cyclic peptides, natural amino acids, unnatural amino acids, and peptidomimetic compounds. Examples of such compounds include T22 (Murakami, T., et al., J. Exp. Med. (1997) 186:1389-1393); T134 (Arakaki, R., et al., J. Virol. (1999) 73:1719-1723; T140 (Tamamura, H., et al., Biochem. Biophys. Res. Comm. (1998) 253:877-882) and its analogs TC14012 and TN14003 (Tamamura, H., et al., Bioorg. Med. Chem. Lett. (2001) 11:1897-1902; Mori, T., et al., Mol. Cancer. Ther. (2004) 3:29-37; Burger, M., et al., Blood (2005) 106:1824-1830); ALX40-4C (Doranz, B. J., et al., J. Exp. Med. (1997) 186:1395-1400; Donzella, G. A., Nat. Med. (1998) 4:72-77; Doranz, B. J., et al., AIDS Res. Hum. Retrovir. (2001) 17:475-486); RCP168 (Zeng, Z., et al., Mol. Cancer. Ther. (2006) 5:3113-3121); CTCE-0021 (Pelus, L. M., et al., Exp. Hematol. (2005) 33:295-307); CTCE-0214 and CTCE-9908 (Zhong, R., et al., Exp. Hematol. (2004) 32:470-475; Kim, S. Y., et al., AACR Meeting Abstracts (2005) Abstract 256; PCT Pub. Nos. WO 01/76615 and WO 01/85196; U.S. Patent Pub. No. 2007/0160574 and related applications); KRH-1120 (Yamamoto, N., et al., J. AIDS Res. (2000) 2:453-460); KRH-1636 (Ichiyama, K., et al., Proc. Natl. Acad. Sci. USA (2003) 100:4185-4190); KRH-2731/CS-3955 (Murakami T., et al., Abstracts of the 11th Conference on Retroviruses and Opportunistic Infections (2004) Abstract 541; PCT Pub. Nos. WO 06/095542 and WO 02/094261); and CXCR4 antagonists described in PCT Pub. Nos. WO 99/47158; WO 99/50461; WO 00/09152; WO 01/94420; and WO 03/090512.

In certain other embodiments, the CXCR4 antagonist is BKT140, including those CXCR4 antagonists described in U.S. Pat. No. 7,423,007 and U.S. Patent Application Pub. No. 2004/0171552; AVR 118; TG-0054, including those CXCR4 antagonists described in U.S. Pat. No. 7,399,776 and U.S. Patent Pub. Nos. 2006/0160860 and 2008/0058382; MSX-122; or POL-6326/POL-2438/POL-3026, including those CXCR4 antagonists described in PCT Pub. No. WO 2008/104090. In certain embodiments, the antagonist may be an antibody, such as a monoclonal antibody, or immunoreactive fragment thereof. The contents of all the foregoing documents are hereby incorporated herein by reference for all purposes.

Methods to synthesize certain of the CXCR4 antagonists disclosed herein are set forth in the U.S. patents and applications above as well as U.S. Pat. No. 6,489,472, PCT Pub. No. WO 02/026721 and certain other documents mentioned herein, which are incorporated herein by reference. Additional suitable CXCR4 antagonists are set forth in Appendix A.

The compounds of the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2^{nd} ed., London (1988).

Compounds useful in the invention, which are amines, may be administered or prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Typically, at physiological pH, the compounds of the invention will be in the forms of the acid addition salts.

Compounds useful in the invention that are carboxylic acids or otherwise acidic may be administered or prepared in forms of salts formed from inorganic or organic bases that are physiologically compatible. Thus, these compounds may be prepared in the forms of their sodium, potassium, calcium, or magnesium salts as appropriate or may be salts with organic bases such as caffeine or ethylamine. These compounds also may be in the form of metal complexes.

When prepared as purified forms, the compounds may also be crystallized as the hydrates or other solvates. Those forms of the compounds used in the invention that contain chiral centers may be optically pure or may contain a mixture of stereoisomers, including racemic mixtures or mixtures of varying optical purity.

The CXCR4 antagonists may be formulated for administration to animal subject using commonly understood formulation techniques well known in the art. Formulations which are suitable for particular modes of administration and for compounds useful in the invention may be found in Remington's The Science and Practice of Pharmacy, 21^{St} edition, Lippincott Williams & Wilkins, Hagerstown, Md.

The CXCR4 antagonists may be administered by injection, such as by intravenous injection, subcutaneous or intraperitoneal injection, and the like. Additional parenteral routes of administration include intramuscular and intraarticular injection. For intravenous or parenteral administration, the compounds are formulated in suitable liquid form with excipients as required. The compositions may contain liposomes or other suitable carriers. For injection intravenously, the solution is made isotonic using standard preparations such as Hank's solution.

Besides injection, other routes of administration may also be used. The compounds may be formulated into tablets, capsules, syrups, powders, or other suitable forms for administration orally. By using suitable excipients, these compounds may also be administered through the mucosa using suppositories or intranasal sprays. Transdermal administration can also be effected by using suitable penetrants and controlling the rate of release.

The formulation and route of administration chosen will be tailored to the individual subject, the nature of the condition to be treated in the subject, and generally, the judgment of the attending practitioner.

The CXCR4 antagonists may be administered as a single bolus dose, a dose over time, as in intravenous or transdermal administration, or in multiple dosages. Suitable dosage ranges for the CXCR4 antagonists vary according to these considerations, but in general, the compounds are administered in the range of about 0.1 μg/kg-10 mg/kg of body weight; preferably the range is about 1 μg/kg-500 μg/kg up to 1 mg/kg of body weight. For a typical 70-kg human subject, thus, the dosage range is from about 7 μg to about 700 mg, preferably from about 70 μg to about 70 mg. Dosages may be higher when the compounds are administered orally or transdermally as compared to, for example, intravenous administration.

Use of a wide variety of chemotherapeutic agents is contemplated by the present invention. Categories of chemotherapeutic agents useful in the present invention include, but are not limited to, alkylating agents such as mustard gas derivatives, ethylenimines, alkylsulfonates, hydrazines and triazines, nitrosoureas, metal salts, plant alkaloids such as podophyllotoxins, taxanes, vinca alkaloids and camptothecin analogs, anti-tumor antibiotics such as chromomycins, anthracyclines and miscellaneous antibiotics, anti-metabolites such as folic acid antagonists, pyrimidine antagonists, purine antagonists and adenosine deaminase inhibitors, topoisomerase inhibitors such as topoisomerase I inhibitors and topoisomerase II inhibitors, and miscellaneous anticancer agents such as ribonucleotide reductase inhibitors, adrenocorticoid inhibitors, anti-microtubule agents and retinoids.

More specific examples of chemotherapeutic agents useful in the present invention include, but are not limited to: ADRIAMYCIN® (doxorubicin), ELLENCE® (epirubicin), IDAMYCIN® (idarubicin), CERUBIDINE® (daunorubicin), NOVANTRONE® (mitoxantrone), BBR2778 (pixantrone), MUTAMYCIN® (mitomycin), BLENOXANE® (bleomycin), COSMEGEN® (dactinomycin), MITHRACIN® (plicamycin), TAXOTERE® (docetaxel), TAXOL® (paclitaxel), CYTOXAN® (cyclophosphamide), MUSTARGEN® (mechlorethamine), LEUKERAN® (chlorambucil), ALKERAN® (melphalan), IFEX® (ifosfamide), MEXATE® (methotrexate), ADRUCIL® (5-fluorouracil, 5-FU), XELODA® (capecitabine), GEMZAR® (gemcitabine), FUDR (floxuridine), CYTOSAR-U® (cytarabine, Ara-C), CLOLAR® (clofarabine), PURINETHOL® (6-mercaptopurine, 6-MP), THIOGUANINE TABLOID® (6-thioguanine, 6-TG), AZASAN® (azathioprine), ARRANON® (nelarabine), LEUSTATIN® (cladribine), FLUDARA® (fludarabine), NIPENT® (pentostatin), HYDREA® (hydroxyurea), LYSODREN® (mitotane), BUSULFEX® (busulfan), HEXALEN® (altretamine), THIOPLEX® (thiotepa), MATULANE® (procarbazine), DTIC-DOME® (dacarbazine), TEMODAR® (temozolomide), CEENU® (lomustine), BICNU® (carmustine), EMCYT® (estramustine), ZANOSAR® (streptozocin), TARGRETIN® (bexarotene), VESANOID® (tretinoin, ATRA), ACCUTANE® (isotretinoin), AMNOID® (tamibarotene), NAVELBINE® (vinorelbine), ONCOYIN® (vincristine), VELBAN® (vinblastine), ELDISINE® (vindesine), IXEMPRA® (ixabepilone), PLATINOL® (cisplatin), PARAPLATIN® (carboplatin), picoplatin, ELOXATIN® (oxaliplatin), HYCAMTIN® (topotecan), CAMPTOSAR® (irinotecan), AMSIDINE® (amsacrine), VEPESID® (etoposide), VUMON® (teniposide), NOVALDEX® (tamoxifen), DECADRON® (dexamethasone), VELCADE® (bortezomib), TARCEVA® (erlotinib), TYKERB® (lapatinib), ZARNESTRA® (tipifarnib), SARASAR® (lonafarnib), IRESSA® (gefitinib), Flavopiridol (alvocidib), FEMARA® (letrozole), ARIMIDEX® (anastrozole), AROMASIN® (exemestane), THALOMID® (thalidomide), CERTICAN® (everolimus), and many others.

As noted above, AMD3100 and AMD3465 are exemplary antagonists of the CXCR4 chemokine receptor (Gerlach, et al., J. Biol. Chem. (2001) 276:14153-14160; Hatse, S., et al., Biochem. Pharmacol. (2005) 70:752-61). Accordingly, in certain embodiments, AMD3100 and AMD3465 may be used in conjunction with one or more chemotherapeutic agent(s) such as, for example, doxorubicin, epirubicin, cyclophosphamide, methotrexate, 5-fluorouracil, paclitaxel, docetaxel, capecitabine, vinorelbine or gemcitabine, to treat a subject afflicted with breast cancer.

A wide variety of chemotherapeutic protocols may be employed, many of such protocols involving combinations of drugs administered simultaneously or in tandem. Some of the commonly used chemotherapeutic regimens for breast cancer include, but are not limited to, four to six cycles of doxorubicin and cyclophosphamide administered once every three weeks (AC); four to six cycles of epirubicin and cyclophosphamide administered once every three weeks (EC); four to six cycles of docetaxel and cyclophosphamide administered once every three weeks (TC); six cycles of cyclophosphamide, epirubicin and 5-fluorouracil administered once every three weeks (CEF); six cycles of cyclophosphamide, doxorubicin and 5-fluorouracil administered once every three weeks (CAF); six cycles of cyclophosphamide, methotrexate and 5-fluorouracil administered once every four weeks (CMF); six cycles of docetaxel, doxorubicin and cyclophosphamide administered once every three weeks (TAC); and four to six cycles of gemcitabine and paclitaxel administered once every three weeks (GT). In some cases, multiple regimens may be combined for additive effect. For example, the AC regimen may be followed by four cycles of paclitaxel or docetaxel administered once every three weeks or by 12 weekly administrations of a smaller dose of paclitaxel or docetaxel. Similarly, the CEF regimen may be followed by three cycles of docetaxel administered once every three weeks. Alternatively, the CMF regimen may be preceded by four cycles of doxorubicin administered once every three weeks.

In certain embodiments, in addition to chemotherapy, HERCEPTIN® (trastuzumab, Genentech, Inc.), a humanized monoclonal antibody that targets the HER2/neu tyrosine kinase receptor, may be included in the regimen depending on the tumor's HER2/neu status and risk of relapse. Trastuzumab may be administered weekly or once every three weeks for about one year or until disease progression. In certain other embodiments, AVASTIN® (bevacizumab, Genentech, Inc.), a humanized monoclonal antibody that targets human vascular endothelial growth factor A (VEGF-A), may also be included in the therapeutic regiment for HER2-negative tumors. Bevacizumab may be administered biweekly for about one year or until disease progression.

The CXCR4 antagonists of the present invention may be administered at various points in the simultaneous or tandem protocols. In certain embodiments, the CXCR4 antagonist may be administered several hours before the first administration of the chemotherapeutic agent, which is repeated over several days. In certain other embodiments, the CXCR4 antagonist may be administered daily before, during, or after the administration of the chemotherapeutic agent. Various combinations of the foregoing agents may be used in such protocols, and the timing and frequency of CXCR4 administration is subject to routine optimization, within ordinary skill. Dosage levels and mode of administration are interdependent. When given subcutaneously, for example, the dosage levels are in the range of 50 μg/kg-1 mg/kg, preferably 200 μg/kg-500 μg/kg.

In certain embodiments, the present methods may further comprise administration of other mobilizing agents, immunomodulatory agents, or other nutritional or therapeutically beneficial agents. The additional factor(s) may be administered in the same composition, in different compositions but simultaneously, or in a tandem protocol with the administration of the CXCR4 antagonist. Among additional factors that can be included are recombinant G-CSF such as NEUPOGEN® (filgrastim), GRANOCYTE®/NEUTROGIN® (lenograstim) and STEMGEN® (ancestim), a covalent conjugate of recombinant G-CSF such as NEULASTA® (pegfilgrastim), granulocyte-macrophage colony stimulating factor (GM-CSF) such as LEUKINS® (sargramostim) and LEUCOMAX® (molgramostim), interleukin-1 (IL-1), interleukin-3 (IL-3), interleukin-8 (IL-8), PIXY-321 (GM-CSF/IL-3 fusion protein), REVIMID™ (CC-5013), ACTIMID™ (CC-4047), macrophage inflammatory protein, stem cell factor and thrombopoietin. In certain embodiments, the presently disclosed methods further comprise the administration of one or more of antibiotics, vitamins, herbal extracts, anti-inflammatories, nutrients, antipyretics, analgesics, cyclophosphamide and the like.

Subjects that will respond favorably to the method of the invention include medical and veterinary subjects generally, including human patients. Among other subjects for whom the methods of the invention is useful are cats, dogs, large animals, and the like, other than standard research animals such as laboratory mice, rabbits, or rats. In general, any subject afflicted with breast cancer would benefit from the methods of the invention.

In another aspect, the invention is directed to a method for treating a subject afflicted with breast cancer, which comprises administering a therapeutically effective amount of a CXCR4 antagonist. Suitable CXCR4 antagonists include any of those disclosed herein. Preferred CXCR4 antagonists include AMD3100 and AMD3465.

In an additional aspect, the present invention is directed to a pharmaceutical or veterinary composition comprising a CXCR4 antagonist in unit dosage form for use in the methods of the invention. The composition comprises a CXCR4 antagonist, a chemotherapeutic agent and a suitable pharmaceutically or veterinary acceptable excipient.

In an additional aspect, the present invention is directed to a pharmaceutical or veterinary composition comprising a CXCR4 antagonist in unit dosage form for use in the methods of the invention. The composition comprises a CXCR4 antagonist and a suitable pharmaceutically or veterinary acceptable excipient. Preferred CXCR4 antagonists include AMD3100 and AMD3465.

Formulations that are suitable for particular modes of administration and for compounds useful in the invention may be found in Remington's The Science and Practice of Pharmacy, 21^{St} edition, Lippincott Williams & Wilkins, Hagerstown, Md.

In certain embodiments, the pharmaceutical or veterinary composition may comprise a CXCR4 antagonist of formula (1) as set forth above. In some specific embodiments, the pharmaceutical or veterinary composition may comprise 1,1′-[1,4-phenylene-bis-(methylene)-bis-1,4,8,11-tetraazacyclotetradecane (AMD3100) and N-[1,4,8,11-tetraazacyclotetradecanyl-(1,4-phenylene-bis-(methylene)]-2-aminoethyl-2-pyridine (AMD3465).

In certain embodiments, the chemotherapeutic agent may comprise doxorubicin, epirubicin, cyclophosphamide, methotrexate, 5-fluorouracil, paclitaxel, docetaxel, capecitabine, vinorelbine, gemcitabine, or a combination thereof.

In certain specific embodiments, AMD3100 or AMD3465 may be used in combination with doxorubicin, epirubicin, cyclophosphamide, methotrexate, 5-fluorouracil, paclitaxel, docetaxel, capecitabine, vinorelbine, gemcitabine, or a combination thereof.

In certain embodiments, the chemotherapeutic agent may comprise doxorubicin. In other embodiments, the chemotherapeutic agent may comprise cyclophosphamide. In further embodiments, the chemotherapeutic agent may comprise combinations of cyclophosphamide with 5-fluorouracil (FC), cyclophosphamide with doxorubicin (AC), cyclophosphamide with epirubicin (EC), cyclophosphamide with docetaxel (TC), gemcitabine with paclitaxel (GT), cyclophosphamide with 5-fluorouracil and doxorubicin (CAF), cyclophosphamide with 5-fluorouracil and epirubicin (CEF), cyclophosphamide with 5-fluorouracil and methotrexate (CMF), or cyclophosphamide with doxorubicin and docetaxel (TAC).

Having now generally described the invention, the same will be more readily understood through reference to the following examples which are provided by way of illustration, and are not intended to be limiting of the present invention, unless specified.

### Example 1

### Efficacy of AMD3465 in MDA-MB-231+MSC Breast Cancer Model

The in vivo therapeutic efficacy of the CXCR4 antagonist AMD3465 was studied in the MDA-MB-231 mouse model of breast cancer. MDA-MB-231 (available from the American Tissue and Cell Collection, Manassas, Va.) is a well-characterized metastatic human breast cancer cell line that has an invasive phenotype in vitro and forms mammary fat pad tumors in vivo. The MSC cells provide stromal support to the MDA-MB-231 cells and enhance tumor growth and metastasis via SDF-1α/CXCL12 and RANTES pathways.

Four groups of 6- to 8-week-old severe combined immunodeficient (SCID) mice (7 animals each) were injected subcutaneously with 1×10⁶ MDA-MB-231 cells admixed with 1×10⁶ human mesenchymal stem (MSC) cells. Starting 48 hours after the injection, Groups 3 and 4 were administered intraperitoneal injections of AMD3465 three times a week (Monday, Wednesday and Friday) for six weeks at 10 mg/kg or 5 mg/kg body weight, respectively. Group 2 was given intraperitoneal injections of anti-human SDF-1α/CXCL12 monoclonal antibody three times a week (Monday, Wednesday and Friday) for six weeks at 100 μg per animal. Group 1 (control) received phosphate-buffered saline (PBS) instead of AMD3465.

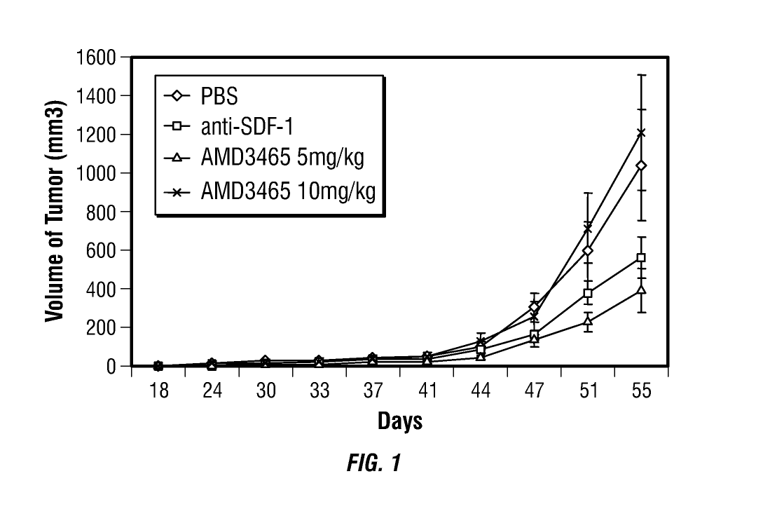

The experimental setup is briefly summarized in Table 1. The size of the tumors was measured approximately every four days beginning on day 18 post-injection. Animals were euthanized when the tumor volume reached approximately 1000 mm³. The time course of the average tumor volume is plotted for each experimental group in FIG. 1.

TABLE 1

| Group | | Animals |
|---|---|---|
| # | Treatment Group | per group |
| 1 | 1 × 10⁶ MDA-MB-231 + 1 × 10⁶ MSC cells, | 7 |
| | control | |
| 2 | 1 × 10⁶ MDA-MB-231 + 1 × 10⁶ MSC cells, | 7 |
| | anti-human SDF-1α/CXCL12 mAb at 100 | |
| | μg/animal MWF for six weeks beginning 48 hours | |
| | post-injection | |
| 3 | 1 × 10⁶ MDA-MB-231 + 1 × 10⁶ MSC cells, | 7 |
| | AMD3465 at 10 mg/kg MWF for six weeks | |
| | beginning 48 hours post-injection | |
| 4 | 1 × 10⁶ MDA-MB-231 + 1 × 10⁶ MSC cells, | 7 |
| | AMD3465 at 5 mg/kg MWF for six weeks | |
| | beginning 48 hours post-injection | |

As shown in FIG. 1, AMD3465 significantly (p=0.07) decreased the rate of tumor growth in the MDA-MB-231/MSC breast cancer model at 5 mg/kg but not at 10 mg/kg. The inhibitory effect of 5 mg/kg AMD3465 was comparable to that of the anti-human SDF-1α/CXCL12 monoclonal antibody.

### Example 2

### Effect of MSC Cells on Efficacy of AMD3465 in MDA-MB-231 Breast Cancer Model

The effect of MSC cells on the in vivo therapeutic efficacy of AMD3465 was studied in the MDA-MB-231 mouse model of breast cancer substantially as described in Example 1.

Six groups of 6- to 8-week-old SCID mice (6 animals each) were injected subcutaneously with 1×10⁶ MDA-MB-231 cells. Groups 4-6 were further injected with 1×10⁶ MSC cells admixed with the MDA-MB-231 cells. Beginning 7 days after the injection, Groups 2 and 5 were administered intraperitoneal injections of AMD3465 three times a week (Monday, Wednesday and Friday) for the duration of the study at 5 mg/kg body weight. Groups 3 and 6 were given intraperitoneal injections of AMD3465 three times a week (Monday, Wednesday and Friday) for the duration of the study at 10 mg/kg body weight. Groups 1 and 4 (controls) received PBS instead of AMD3465.

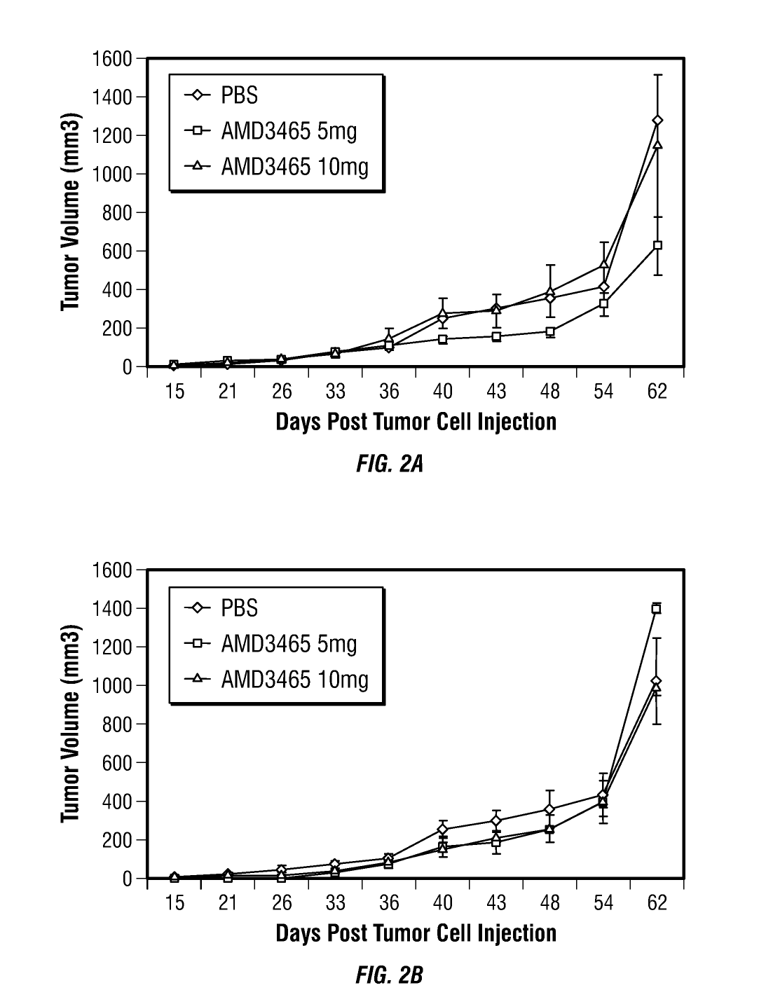

The experimental setup is briefly summarized in Table 2. The size of the tumors was measured approximately every five days, beginning on day 15 post-injection. Animals were euthanized when the tumor volume reached approximately 1000 mm³. The time course of the average tumor volume is plotted for each experimental group in FIGS. 2A and 2B.

TABLE 2

| Group | | Animals |
|---|---|---|
| # | Treatment Group | per group |
| 1 | 1 × 10⁶ MDA-MB-231, control | 6 |
| 2 | 1 × 10⁶ MDA-MB-231, AMD3465 at 5 mg/kg | 6 |
| | MWF for duration of study beginning 7 days post- | |
| | injection | |
| 3 | 1 × 10⁶ MDA-MB-231, AMD3465 at 10 mg/kg | 6 |
| | MWF for duration of study beginning 7 days post- | |
| | injection | |
| 4 | 1 × 10⁶ MDA-MB-231 + 1 × 10⁶ MSC cells, | 6 |
| | Control | |
| 5 | 1 × 10⁶ MDA-MB-231 + 1 × 10⁶ MSC cells, | 6 |
| | AMD3465 at 5 mg/kg MWF for duration of study | |
| | beginning 7 days post-injection | |
| 6 | 1 × 10⁶ MDA-MB-231 + 1 × 10⁶ MSC cells, | 6 |
| | AMD3465 at 10 mg/kg MWF for duration of study | |
| | beginning 7 days post-injection | |

As shown in FIG. 2A, AMD3465 significantly (p=0.024) decreased the rate of tumor growth in the MDA-MB-231 breast cancer model at 5 mg/kg but not at 10 mg/kg in the presence of MSC cells, which is consistent with the result in Example 1. In contrast, as shown in FIG. 2B, AMD3465 had no statistically significant effect on the rate of tumor growth in the absence of MSC cells. This result appears to suggest that 5 mg/kg AMD3465 exerts its effect at the interface between the breast cancer cells and the stromal cells.

### Example 3

### Efficacy of AMD3465 in Established MDA-MB-231 Tumors in Absence of MSC Cells

The in vivo therapeutic efficacy of AMD3465 was studied in established MDA-MB-231 tumors in the absence of stromal cells substantially as described above.

Four groups of 6- to 8-week-old SCID mice (8 animals each) were injected subcutaneously with 1×10⁶ MDA-MB-231 cells. At the start of dosing (about 21 days post-injection), the tumor volume was approximately 100 mm³. Beginning approximately 21 days after the injection, Groups 2-4 were administered intraperitoneal injections of AMD3465 three times a week (Monday, Wednesday and Friday) for the duration of the study at 2.5 mg/kg, 5 mg/kg and 10 mg/kg body weight, respectively. Group 1 (control) received PBS instead of AMD3465.

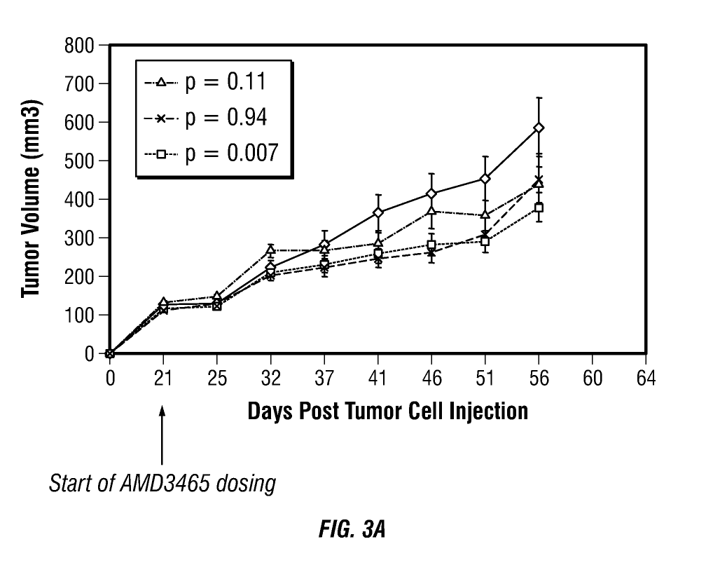

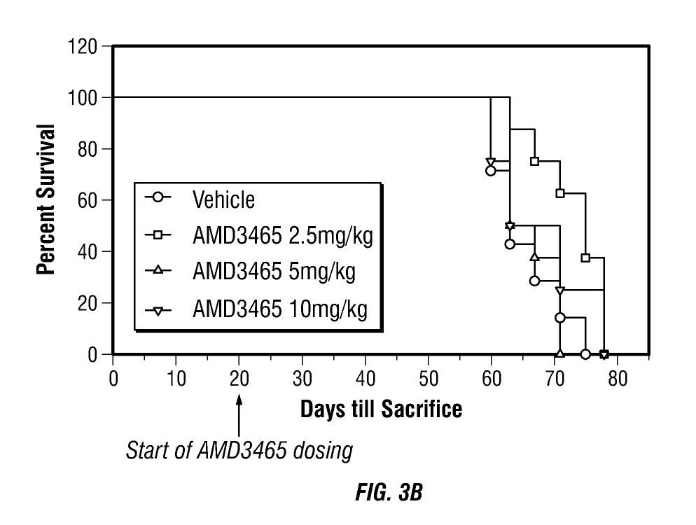

The experimental setup is briefly summarized in Table 3. The size of the tumors was measured approximately every five days, beginning on day 21 post-injection. Animals were euthanized when the tumor volume reached approximately 1000 mm³. The time course of the average tumor volume is plotted for each experimental group in FIG. 3A. The mean survival in each group was estimated by the Kaplan-Meier method, as shown in FIG. 3B.

TABLE 3

| Group | | Animals |
|---|---|---|
| # | Treatment Group | per group |
| 1 | 1 × 10⁶ MDA-MB-231, control | 8 |
| 2 | 1 × 10⁶ MDA-MB-231, AMD3465 at 2.5 mg/kg | 8 |
| | MWF for duration of study beginning 21 days post- | |
| | injection | |
| 3 | 1 × 10⁶ MDA-MB-231, AMD3465 at 5 mg/kg | 8 |
| | MWF for duration of study beginning 21 days post- | |
| | injection | |
| 4 | 1 × 10⁶ MDA-MB-231, AMD3465 at 10 mg/kg | 8 |
| | MWF for duration of study beginning 21 days post- | |
| | injection | |

As shown in FIG. 3A, AMD3465 significantly decreased the rate of tumor growth in established MDA-MB-231 tumors at 2.5 mg/kg (p=0.007) but not at 5 or 10 mg/kg body weight in the absence of MSC cells, which is consistent with the result in Example 2. As shown in FIG. 3B, AMD3465 also increased the mean survival at 2.5 mg/kg but not at 5 or 10 mg/kg body weight in the absence of MSC cells. It is worth noting, however, that despite a clear trend, no statistical significance was achieved at 2.5 mg/kg (p=0.25).

### Example 4

### Efficacy of AMD3100 in Established Syngeneic 4T1 Tumors

The in vivo therapeutic efficacy of AMD3100 was studied in established 4T1 tumors. The 4T1 cell line (available from the American Tissue and Cell Collection, Manassas, Va.) was derived from a spontaneously arising BALB/c mammary tumor and is commonly used as a syngeneic mouse model of metastatic breast cancer. When introduced orthotopically, the 4T1 line grows rapidly at the primary site and forms metastases in lungs, liver, bone and brain over a period of 3-6 weeks. When introduced intravenously or arterially, metastases are apparent in the same organs after 1-2 weeks. The rapid and efficient metastasis to organs affected in human breast cancer makes the 4T1 model an excellent mouse model for the study of metastatic progression of breast cancer in humans.

Three groups of 6- to 8-week-old SCID mice (6 animals each) were injected subcutaneously with 2×10⁵ 4T1 cells. At the start of dosing (about 14 days post-injection), the tumor volume was approximately 100 mm³. Beginning approximately 21 days post-injection, Groups 2 and 3 were administered subcutaneous injections of AMD3100 three times a week (Monday, Wednesday and Friday) for two weeks at 1.25 and 2.5 mg/kg body weight, respectively. Group 1 (control) received PBS instead of AMD3100.

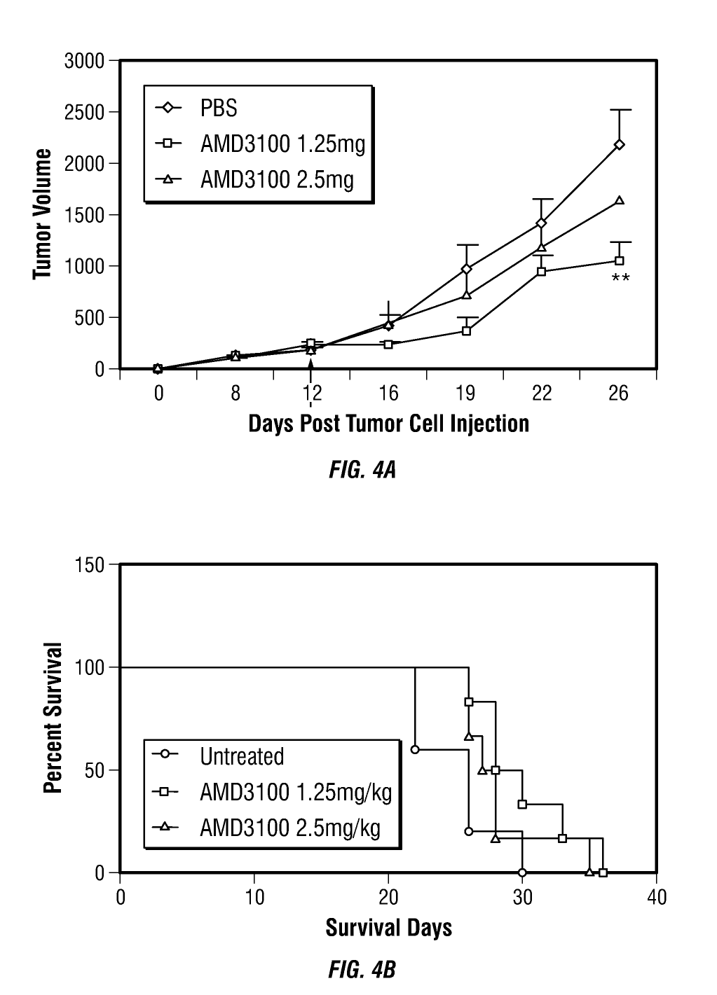

The experimental setup is briefly summarized in Table 4. The size of the tumors was measured approximately every four days, beginning on day 10 post-injection. Animals were euthanized when the tumor volume reached approximately 1000 mm³. This study was terminated early due to severe ulceration of the tumors. The time course of the average tumor volume is plotted for each experimental group in FIG. 4A. The mean survival in each group was estimated by the Kaplan-Meier method, as shown in FIG. 4B.

TABLE 4

| Group | | Animals |
|---|---|---|
| # | Treatment Group | per group |
| 1 | 2 × 10⁵ 4T1, control | 6 |
| 2 | 2 × 10⁵ 4T1, AMD3100 at 1.25 mg/kg MWF for | 6 |
| | two weeks beginning 14 days post-injection | |
| 3 | 2 × 10⁵ 4T1, AMD3100 at 2.5 mg/kg MWF for | 6 |
| | two weeks beginning 14 days post-injection | |

As shown in FIG. 4A, AMD3100 significantly decreased the rate of tumor growth in established 4T1 tumors at 1.25 mg/kg but not at 2.5 mg/kg body weight. The onset of tumor growth was delayed by approximately one week, and the growth appeared to have largely leveled off by day 26 post-injection. As shown in FIG. 4B, AMD3100 also increased the mean survival at 1.25 mg/kg but not at 2.5 mg/kg body weight in the 4T1 syngeneic breast cancer model. However, no statistical significance was achieved at either concentration of AMD3100 tested (p=0.1 at 1.25 mg/kg; p=0.4 at 2.5 mg/kg).

### Example 5

### AMD3100-Mediated Chemosensitization in MDA-MB-231 Tumors

The in vivo therapeutic efficacy of the CXCR4 antagonist AMD3100 was studied in combination with the chemotherapeutic agent doxorubicin in the MDA-MB-231 breast cancer model substantially as described in Examples 1-3 above.

Four groups of 6- to 8-week-old SCID mice (6 animals each) were injected subcutaneously with 1×10⁶ MDA-MB-231 cells. At the start of dosing (about 28 days post-injection), the tumor volume was approximately 100 mm³. Beginning approximately 28 days after the injection, Groups 2-4 were administered intraperitoneal injections of doxorubicin once a week (Monday) for three weeks at 2 mg/kg body weight. Additionally, Groups 3 and 4 were given subcutaneous injections of AMD3100 three times a week (Monday, Wednesday and Friday) for three weeks at 1.25 and 2.5 mg/kg body weight, respectively. Group 1 (control) received PBS instead of doxorubicin and/or AMD3100.

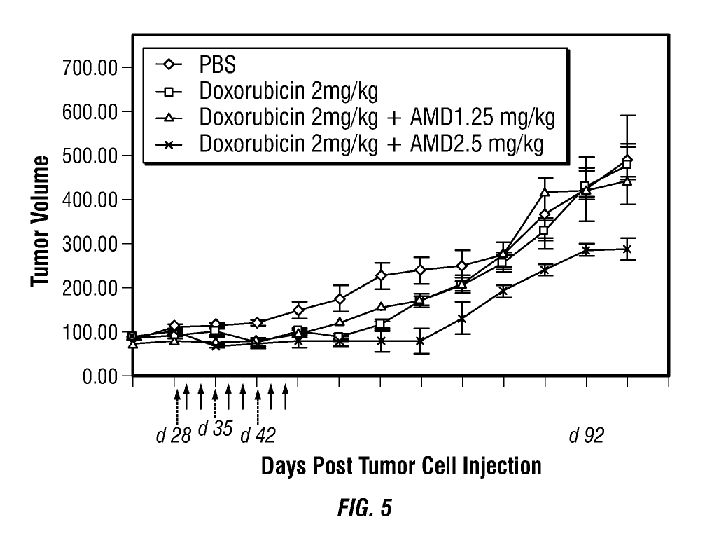

The experimental setup is briefly summarized in Table 5. The size of the tumors was measured approximately every six days, beginning on day 21 post-injection. Animals were euthanized when the tumor volume reached approximately 1000 mm³. The time course of the average tumor volume is plotted for each experimental group in FIG. 5.

TABLE 5

| Group | | Animals |
|---|---|---|
| # | Treatment Group | per group |
| 1 | 1 × 10⁶ MDA-MB-231, control | 6 |
| 2 | 1 × 10⁶ MDA-MB-231, doxorubicin at 2 mg/kg | 6 |
| | weekly (M) for three weeks beginning 28 days post- | |
| | injection | |
| 3 | 1 × 10⁶ MDA-MB-231, doxorubicin at 2 mg/kg | 6 |
| | weekly (M) for three weeks beginning 28 days post- | |
| | injection + AMD3100 at 1.25 mg/kg MWF for | |
| | three weeks beginning 28 days post-injection | |
| 4 | 1 × 10⁶ MDA-MB-231, doxorubicin at 2 mg/kg | 6 |
| | weekly (M) for three weeks beginning 28 days post- | |
| | injection + AMD3100 at 2.5 mg/kg MWF for | |
| | three weeks beginning 28 days post-injection | |

As shown in FIG. 5, AMD3100 potentiated the effect of 2 mg/kg doxorubicin on the tumor growth at 2.5 mg/kg but not at 1.25 mg/kg body weight. The onset of tumor growth was delayed by several weeks, and the growth appeared to have leveled off by day 92 post-injection in animals treated with doxorubicin and 2.5 mg/kg AMD3100. The potentiating effect appeared to be statistically significant at day 62 of the study (p=0.01), although overall statistical significance was not achieved (p=0.25).

### Example 6

### AMD3100-Mediated Chemosensitization in MDA-MB-231 Tumors—Alternative Dosing

The in vivo therapeutic efficacy of the CXCR4 antagonist AMD3100 was studied in combination with the chemotherapeutic agent doxorubicin in the MDA-MB-231 breast cancer model substantially as described above in Example 5, with the exceptions that twice as many cells were injected, a lower dose of doxorubicin was used, and both drugs were administered for the entire duration of the study instead of the first three weeks.

Three groups of 6- to 8-week-old SCID mice (6 animals each) were injected subcutaneously with 2×10⁶ MDA-MB-231 cells. At the start of dosing (about 14 days post-injection), the tumor volume was approximately 100 mm³. Beginning approximately 14 days after the injection, Groups 2 and 3 were administered intraperitoneal injections of doxorubicin once a week (Monday) for the duration of the study at 1 mg/kg body weight. Additionally, Group 3 was given subcutaneous injections of AMD3100 three times a week (Monday, Wednesday and Friday) for the duration of the study at 2.5 mg/kg body weight. Group 1 (control) received PBS instead of doxorubicin and/or AMD3100.

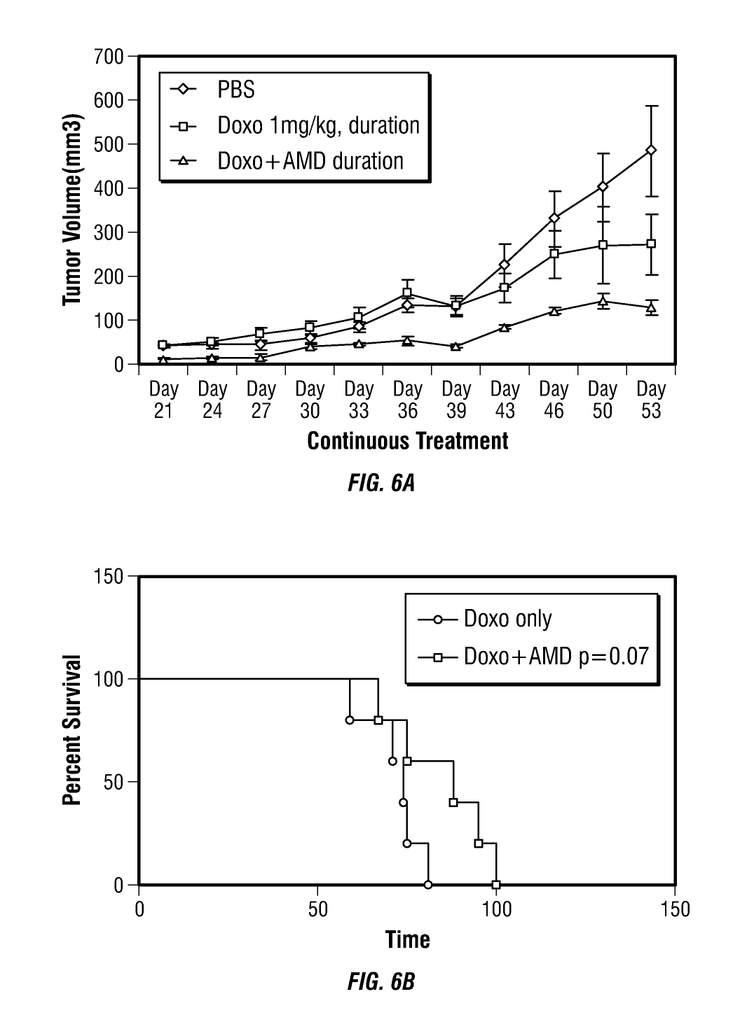

The experimental setup is briefly summarized in Table 6. The size of the tumors was measured approximately every three days, beginning on day 21 post-injection. Animals were euthanized when the tumor volume reached approximately 1000 mm³. The time course of the average tumor volume is plotted for each experimental group in FIG. 6A. The mean survival in each group was estimated by the Kaplan-Meier method, as shown in FIG. 6B.

TABLE 6

| Group | | Animals |
|---|---|---|
| # | Treatment Group | per group |
| 1 | 2 × 10⁶ MDA-MB-231, control | 6 |
| 2 | 2 × 10⁶ MDA-MB-231, doxorubicin at 1 mg/kg | 6 |
| | weekly (M) for duration of study beginning 14 days | |
| | post-injection | |
| 3 | 2 × 10⁶ MDA-MB-231, doxorubicin at 1 mg/kg | 6 |
| | weekly (M) for duration of study beginning 14 days | |
| | post-injection + AMD3100 at 2.5 mg/kg MWF | |
| | for duration of study beginning 14 days post- | |
| | injection | |

As shown in FIG. 6A, AMD3100 significantly potentiated the effect of 1 mg/kg doxorubicin on the tumor growth at 2.5 mg/kg body weight. In animals treated with doxorubicin and AMD3100, the onset of tumor growth was delayed by about two weeks, the rate of growth was significantly (p=0.04) decreased compared to doxorubicin alone and appeared to have leveled off by day 50 post-injection. As shown in FIG. 6B, the combination of 2.5 mg/kg AMD3100 and 1 mg/kg doxorubicin also significantly (p=0.07) increased the mean survival compared to doxorubicin alone.

The in vivo results described in Examples 1-6 strongly suggest that there is a potential role for CXCR4 antagonists in combination with chemotherapeutic agents in the treatment of breast cancer.

### APPENDIX A

Exemplary CXCR4 antagonists of Formula I include compounds of formula (1A):

V—CR₂—Ar¹—CR₂NR—(CR₂)ₓ—Ar² (1A)
wherein V is a substituted heterocycle of 9-24 members containing 2-4 optionally substituted amine nitrogen atoms spaced from each other by 2 or more optionally substituted carbon atoms, and which heterocycle may optionally comprise a fused aromatic or heteroaromatic ring, and wherein
(a) said heterocycle contains at least one O or S, said O or S spaced from any adjacent heteroatom by at least 2 carbon atoms, and wherein said S is optionally oxidized or
(b) at least one carbon atom in said ring is substituted by an electron-withdrawing substituent, or
(c) both (a) and (b);
and wherein each R is independently H or a straight chain, branched or cyclic alkyl containing 1-6C;
x is 0-4;
Ar¹ is an unsubstituted or substituted aromatic or heteroaromatic moiety; and
Ar² is an unsubstituted or substituted aromatic or heterocyclic group.

In another embodiment of Formula 1, the CXCR4 antagonist has formula

V—CH₂—Ar¹—CH₂NR—CH₂—Ar²
wherein V is a heterocycle as defined in formula (1A), and wherein:
(a) said heterocycle is substituted with halo or ═O; or
(b) said heterocycle contains O or S; or
(c) both (a) and (b),
and wherein Ar¹ is unsubstituted 1, 3 or 1,4-phenylene, R is H, methyl or ethyl and Ar² is unsubstituted phenyl or pyridinyl. Preferred embodiments of x are 0-2 and 1-2.

The heterocycle V may contain 3 N and at least one carbon atom in the heterocycle that is substituted by at least one fluoro substituent. The R moiety may independently be hydrogen or methyl. The number of (CR₂)ₓ groups may be 0-4, 0-2, or 1-2. The Ar¹ moiety may be 1, 3 or 1,4-phenylene. The Ar² moiety may be phenyl or pyridyl. The heterocycle V may be a 12-16 membered heterocycle, or may contain O or S as a ring member. The heterocycle V may also contain an oxidized sulfur as a ring member. In one example, at least one carbon in the heterocycle V is substituted by ═O.

Compounds of formula (1A) and methods of synthesizing such compounds are described in PCT Pub. No. WO 01/44229 and U.S. Pat. Nos. 6,667,320 and 7,022,717, incorporated herein by reference.

Related to these compounds having formula (1B):

V—CR¹R²—Ar—CR³R⁴—N(R⁵)—(CR⁶R⁷)ₓ—R⁸ (1B)
wherein V is an optionally substituted 1,4,8,11-tetraazacyclotetra-decanyl, 4,7,10,17-tetraazabicyclo[13.3.1]heptadeca-1(17),13,15-trienyl, 1,4,7-triazacyclotetra-decanyl, 4,7,10-triazabicyclo[13.3.1]heptadeca-1(17),13,15-trienyl, 1,7-diazacyclotetradecanyl, or 4,10-diazabicyclo[13.31.1]heptadeca-1(17),13,15-trienyl system;
R¹ to R⁷ may be the same or different and are independently selected from hydrogen or straight, branched or cyclic C₁₋₆ alkyl;
R⁸ is pyridyl, pyrimidinyl, pyrazinyl, imidazolyl, thiophene-yl, thiophenyl, aminobenzyl, piperidinyl, purine, piperazinyl, phenylpiperazinyl, or mercaptan;
Ar is a phenylene ring optionally substituted at single or multiple positions with alkyl, aryl, amino, alkoxy, hydroxy, halogen, carboxyl and/or carboxamido; and
x is 1 or 2.

In the above formula (1B), the V moiety may be optionally substituted by hydroxyl, alkoxy, thiol, thioalkyl, halogen, nitro, carboxy, amido, sulfonic acid, and/or phosphate.

Compounds of formula (1B), pharmaceutically acceptable salts or metal complexes thereof, and methods of synthesizing such compounds are described in PCT Pub. No. WO 00/02870 and U.S. Pat. No. 5,817,807, incorporated herein by reference.

Other CXCR4 antagonists are of formula (1C):

V²—CR₉R₁₀—Ar² (1C)
wherein V² is an optionally substituted 1,4,8,11-tetraazacyclotetra-decanyl or 4,7,10,17-tetraazabicyclo[13.3.1]heptadeca-1(17),13,15-trienyl system;
R₉ and R₁₀ may be the same or different and are independently selected from hydrogen or straight, branched or cyclic C₁₋₆ alkyl;
Ar² is an aromatic or heterocyclic ring each optionally substituted at single or multiple positions with electron-donating or withdrawing groups and/or aromatic and heterocyclic groups and their alkyl derivatives thereof, and the acid addition salts and metal complexes.

In the above formula (1C), Ar₂ may be optionally substituted with alkyl, aryl, amino, alkoxy, hydroxy, halogen, carboxyl and/or carboxamido. In particular examples, Ar₂ is optionally substituted with alkoxy, alkyl, or halogen.

Compounds having formula (1C), and methods of synthesizing the same, are described in PCT Pub. No. WO 00/02870 and U.S. Pat. Nos. 6,506,770; 6,756,391; 7,160,872; 6,872,714; and 7,414,065, incorporated herein by reference.

Other CXCR4 antagonists are of formula (1D):

V—R-A-R′—W (1D)
wherein V and W are independently cyclic polyamine moieties having from 9 to 32 ring members and from 3 to 8 amine nitrogens in the ring spaced by 2 or more carbon atoms from each other, and having one or more aromatic or heteroaromatic rings fused thereto,
A is an aromatic or heteroaromatic moiety when V and W have one or more aromatic or heteroaromatic moieties fused thereto, with or without an additional heteroatom other than nitrogen incorporated in the ring, or A is an aromatic or heteroaromatic moiety when V and W contain a heteroatom other than nitrogen incorporated in the ring without having one or more aromatic or heteroaromatic moieties fused thereto,
and R and R′ are each a substituted or unsubstituted alkylene chain or heteroatom-containing chain which spaces the cyclic polyamines and the moiety A.

In the above Formula (1D), R and R′ may each be methylene. In one example, A is 1,3- or 1,4-phenylene. In another example, each V and W is an unsubstituted or substituted tricyclic or bicyclic ring system containing only carbon and nitrogen atoms in the rings. One of the cyclic ring systems may be a 10 to 20 membered polyamine ring system having from 3 to 6 amine nitrogen atoms, and the ring system or systems is a fused benzyl or pyridinyl ring system.

Compounds having formula (1D), and methods of synthesizing such compounds, are described in U.S. Pat. No. 5,698,546, incorporated herein by reference.

Other CXCR4 antagonists are of formula (1E):

Z—R-A-R′—Y (1E)
where Z and Y are identical cyclic polyamine moieties having from 10 to 15 ring members and from 3 to 6 amine nitrogens in the ring spaced by 2 or more carbon atoms from each other, said amine nitrogens being the only ring heteroatoms,
A is an aromatic or heteroaromatic moiety other than quinoline,
R and R′ are each methylene linked to nitrogen atoms in Z and Y, the amine nitrogen atoms being otherwise unsubstituted.

In the above formula (1E), each moiety Z and Y may have 14 ring members and 4 amine nitrogens in the ring. Compounds having formula (1E), and methods of synthesizing such compounds, are described in U.S. Pat. No. 5,583,131, incorporated herein by reference.

The CXCR4 antagonist may be of formula (1F):

Z-(A)ₙ-Y (1F)
where Z and Y are independently cyclic polyamine moieties having from 9 to 32 ring members and from 3 to 8 amine nitrogen atoms in the ring,
A is a linking atom or group, and n is O or an integer from 1 to 6.

In the above formula (1F) each Z and Y moiety may have 10 to 24 ring members, or 12 to 18 ring members. Each Z and Y moiety may also have 4 to 6 amine nitrogen atoms in the ring. In one example, n is 0. In another example, A is methylene.

Compounds having formula (1F), and methods of synthesizing such compounds, are described in U.S. Pat. Nos. 5,021,409 and 6,001,826, incorporated herein by reference.

In specific embodiments, the compound of formula (1) is selected from:
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
methylene (or polymethylene) di 1-N-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylene-bis(methylene)]-bis-1,4,8,11-tetraazacyclotetradecane;
1,1′-[1,4-phenylene-bis(methylene)]-bis-1,4,8,11-tetraazacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenyleneis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.18,12]tetracosa-1(23),8,10,12(24),19,21-hexaene;
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine;
N-[1,4,8,11-tetraazacyclotetradecanyl-1,4-phenylenebis(methylene)]-4-(amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine;
N-[1,4,8,11-tetraazacyclotetradecanyl-1,4-phenylenebis(methylene)]-2-(amino-ethyl)pyridine;
N-[1,4,8,11-tetraazacyclotetradecanyl-1,4-phenylenebis(methylene)]-2-(amino-methyl)thiophene;
N-[1,4,8,11-tetraazacyclotetradecanyl-1,4-phenylenebis(methylene)]-2-(amino-ethyl)mercaptan;
N-[1,4,8,11-tetraazacyclotetradecanyl-1,4-phenylenebis(methylene)]-2-amino-benzylamine;
N-[1,4,8,11-tetraazacyclotetradecanyl-1,4-phenylenebis(methylene)]-4-amino-benzylamine;
N-[1,4,8,11-tetraazacyclotetradecanyl-1,4-phenylenebis(methylene)]-4-(amino-ethyl)imidazole;
N-[1,4,8,11-tetraazacyclotetradecanyl-1,4-phenylenebis(methylene)]-benzylamine;
N-[1,4,8,11-tetraazacyclotetradecanyl-1,4-phenylenebis(methylene)]-purine;
N-[1,4,8,11-tetraazacyclotetradecanyl-1,4-phenylenebis(methylene)]-4-phenylpiperazine;
1-[2,6-dimethoxypyrid-4-yl(methylene)]-1,4,8,11-tetraazacyclotetradecane;
1-[2-chloropyrid-4-yl(methylene)]-1,4,8,11-tetraazacyclotetradecane;
1-[2,6-dimethylpyrid-4-yl(methylene)]-1,4,8,11-tetraazacyclotetradecane;
1-[2-methylpyrid-4-yl(methylene)]-1,4,8,11-tetraazacyclotetradecane;
1-[2,6-dichloropyrid-4-yl(methylene)]-1,4,8,11-tetraazacyclotetradecane;
1-[2-chloropyrid-5-yl(methylene)]-1,4,8,11-tetraazacyclotetradecane;
7-[4-methylphenyl(methylene)]-4,7,10,17-tetraazabicyclo[13.3.1]heptadeca-1(17),13,15-triene;
N-[4-(1,4,7-triazacyclotetra-decanyl)-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17),13,15-trienyl)-1,4-phenylenebis(methylene)]-2-(aminomethyl)pyridine;
N-[7-(4,7,10-triazabicyclo[13.3.1]heptadeca-1(17),13,15-trienyl)-1,4-phenylenebis(methylene)]-2-(aminomethyl)pyridine;
N-[4-[4,7,10-triazabicyclo[13.3.1]heptadeca-1(17),13,15-trienyl]-1,4-phenylenebis(methylene)]-2-(aminomethyl)pyridine;
N4-[4,7,10,17-tetraazabicyclo[13.3.1]heptadeca-1(17),13,15-trienyl]-1,4-phenylenebis(methylene)]-2-(aminomethyl)pyridine;
N-[3-(3,6,17-triazabicyclo[13.3.1]heptadeca-1(17),13,15-trienyl)-1,4-phenylenebis(methylene)]-2-(aminomethyl)pyridine;
N-[3-(3,6,17-triazabicyclo[13.3.1]heptadeca-1(17),13,15-trienyl)-1,3-phenylenebis(methylene)]-2-(aminomethyl)pyridine;
N-[4-(4,7,17-triazabicyclo[13.3.1]heptadeca-1(17),13,15-trienyl)-1,4-phenylenebis(methylene)]-2-(aminomethyl)pyridine;
N-[7-(4,7,17-triazabicyclo[13.3.1]heptadeca-1(17),13,15-trienyl)-1,4-phenylenebis(methylene)]-2-(aminomethyl)pyridine;
N-[6-(3,6,9-triazabicyclo[11.3.1]pentadeca-1(15),11,13-trienyl)-1,3-phenylenebis(methylene)]-2-(aminomethyl)pyridine;
N-[4-(1,7-diazacyclotetradecanyl)-1,4-phenylenebis(methylene)]-2-(aminomethyl)pyridine;
N-[7-(4,10-diazabicyclo[13.3.1]heptadeca-1(17),13,15-trienyl)-1,4-phenylenebis(methylene)]-2-(aminomethyl)pyridine;
N-[7-(4,10,17-triazabicyclo[13.3.1]heptadeca-1(17),13,15-trienyl)-1,4-phenylenebis(methylene)]-2-(aminomethyl)pyridine;
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-onyl)-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,4,7-triazacyclotetradecanyl)-1,4-phenylenebis(methylene)]-2-(aminomethyl)pyridine;
N-[4-(11-thia-1,4,7-triazacyclotetradecanyl)-1,4-phenylenebis(methylene)]-2-(aminomethyl)pyridine;
N-[4-(11-sulfoxo-1,4,7-triazacyclotetradecanyl)-1,4-phenylenebis(methylene)]-2-(aminomethyl)pyridine;
N-[4-(11-sulfono-1,4,7-triazacyclotetradecanyl)-1,4-phenylenebis(methylene)]-2-(aminomethyl)pyridine; and
N-[4-(3-carboxo-1,4,7-triazacyclotetradecanyl)-1,4-phenylenebis(methylene)]-2-(aminomethyl)pyridine;
   or a pharmaceutically acceptable salt thereof.

It is understood that the foregoing detailed description and accompanying examples are merely illustrative, and are not to be taken as limitations upon the scope of the invention. U.S. patents and publications referenced herein are incorporated by reference.

1. A method for treating breast cancer in a human comprising administering to the human a therapeutically effective amount of a CXCR4 antagonist, wherein the CXCR4 antagonist comprising is a compound of the formula:
Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula
—N(R)—(CR₂)ₙ—X
wherein each R is independently H or straight, branched or cyclic alkyl (1-6C), n is 1 or 2, and X is an aromatic ring, including heteroaromatic rings, or is a mercaptan,
or Z′ may be of the formula
—Ar(Y)ⱼ
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

2. The method of claim 1, wherein Z and Z′ are both cyclic polyamines.

3. The method of claim 1, wherein Z and Z′ are identical.

4. The method of claim 1, wherein Z is a cyclic polyamine that contains 10-24 members and contains 4 nitrogen atoms.

5. The method of claim 1, wherein Z and Z′ are both 1,4,8,11-tetraazocyclotetradecane.

6. The method of claim 1, wherein the linker comprises an aromatic ring bracketed by two methylene moieties.

7. The method of claim 6, wherein the linker is 1,4-phenylene-bis-methylene.

8. The method of claim 7, wherein the compound of formula (1) is 1,1′-[1,4-phenylene-bis-(methylene)-bis-1,4,8,11-tetraazacyclotetradecane.

9. The method of claim 1, wherein formula (1) is in the form of an acid addition salt.

10. The method of claim 9, wherein the acid addition salt is hydrochloride.

11. The method of claim 1, wherein Z′ is of the formula
—N(R)—(CR₂)ₙ—X
wherein each R, N and X are as defined in claim 1.

12. The method of claim 11, wherein the linker comprises an aromatic ring bracketed by two methylene moieties.

13. The method of claim 12, wherein the linker is 1,4-phenylene-bis-methylene.

14. The method of claim 11, wherein each R is H, n is 2 and X is substituted or unsubstituted pyridyl.

15. The method of claim 11, wherein Z′ is 2-aminomethyl-pyridine.

16. The method of claim 15, wherein the compound of formula (1) is N-[1,4,8,11-tetraazacyclotetradecanyl-(1,4-phenylene-bis-(methylene)]-2-aminoethyl-2-pyridine.

17. The method of claim 1, wherein the compound of formula (1) is selected from the group consisting of:
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
methylene (or polymethylene) di 1-N-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′(1,2-butanediyl)bis-1,4,8,11-tetraazacyclotetradecane;
11,11′(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylene-bis(methylene)]-bis-1,4,8,11-tetraazacyclotetradecane;
1,1-[1,4-phenylene-bis(methylene)]-bis-1,4,8,11-tetraazacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenyleneis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.18,12]tetracosa-1(23),8,10,12(24),19,21-hexaene;
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine;
N-[1,4,8,11-tetraazacyclotetradecanyl-1,4-phenylenebis(methylene)]-4-(amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine;
N-[1,4,8,11-tetraazacyclotetradecanyl-1,4-phenylenebis(methylene)]-2-(amino-ethyl)pyridine;
N-[1,4,8,11-tetraazacyclotetradecanyl-1,4-phenylenebis(methylene)]-2-(amino-methyl)thiophene;
N-[1,4,8,11-tetraazacyclotetradecanyl-1,4-phenylenebis(methylene)]-2-(amino-ethyl)mercaptan;
N-[1,4,8,11-tetraazacyclotetradecanyl-1,4-phenylenebis(methylene)]-2-amino-benzylamine;
N-[1,4,8,11-tetraazacyclotetradecanyl-1,4-phenylenebis(methylene)]-4-amino-benzylamine;
N-[1,4,8,11-tetraazacyclotetradecanyl-1,4-phenylenebis(methylene)]-4-(amino-ethyl)imidazole;
N-[1,4,8,11-tetraazacyclotetradecanyl-1,4-phenylenebis(methylene)]-benzylamine;
N-[1,4,8,11-tetraazacyclotetradecanyl-1,4-phenylenebis(methylene)]-purine;
N-[1,4,8,11-tetraazacyclotetradecanyl-1,4-phenylenebis(methylene)]-4-phenylpiperazine;
1-[2,6-dimethoxypyrid-4-yl(methylene)]-1,4,8,11-tetraazacyclotetradecane;
1-[2-chloropyrid-4-yl(methylene)]-1,4,8,11-tetraazacyclotetradecane;
1-[2,6-dimethylpyrid-4-yl(methylene)]-1,4,8,11-tetraazacyclotetradecane;
1-[2-methylpyrid-4-yl(methylene)]-1,4,8,11-tetraazacyclotetradecane;
1-[2,6-dichloropyrid-4-yl(methylene)]-1,4,8,11-tetraazacyclotetradecane;
1-[2-chloropyrid-5-yl(methylene)]-1,4,8,11-tetraazacyclotetradecane;
7-[4-methylphenyl(methylene)]-4,7,10,17-tetraazabicyclo[13.3.1]heptadeca-1(17),13,15-triene;
N-[4-(1,4,7-triazacyclotetra-decanyl)-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17),13,15-trienyl)-1,4-phenylenebis(methylene)]-2-(aminomethyl)pyridine;
N-[7-(4,7,10-triazabicyclo[13.3.1]heptadeca-1(17),13,15-trienyl)-1,4-phenylenebis(methylene)]-2-(aminomethyl)pyridine;
N-[4-[4,7,10-triazabicyclo[13.3.1]heptadeca-1(17),13,15-trienyl]-1,4-phenylenebis(methylene)]-2-(aminomethyl)pyridine;
N-[4-[4,7,10,17-tetraazabicyclo[13.3.1]heptadeca-1(17),13,15-trienyl]-1,4-phenylenebis(methylene)]-2-(aminomethyl)pyridine;
N-[3-(3,6,17-triazabicyclo[13.3.1]heptadeca-1(17),13,15-trienyl)-1,4-phenylenebis(methylene)]-2-(aminomethyl)pyridine;
N-[3-(3,6,17-triazabicyclo[13.3.1]heptadeca-1(17),13,15-trienyl)-1,3-phenylenebis(methylene)]-2-(aminomethyl)pyridine;
N-[4-(4,7,17-triazabicyclo[13.3.1]heptadeca-1(17),13,15-trienyl)-1,4-phenylenebis(methylene)]-2-(aminomethyl)pyridine;
N-[7-(4,7,17-triazabicyclo[13.3.1]heptadeca-1(17),13,15-trienyl)-1,4-phenylenebis(methylene)]-2-(aminomethyl)pyridine;
N-[6-(3,6,9-triazabicyclo[11.3.1]pentadeca-1(15),11,13-trienyl)-1,3-phenylenebis(methylene)]-2-(aminomethyl)pyridine;
N-[4-(1,7-diazacyclotetradecanyl)-1,4-phenylenebis(methylene)]-2-(aminomethyl)pyridine;
N-[7-(4,10-diazabicyclo[13.3.1]heptadeca-1(17),13,15-trienyl)-1,4-phenylenebis(methylene)]-2-(aminomethyl)pyridine;
N-[7-(4,10,17-triazabicyclo[13.3.1]heptadeca-1(17),13,15-trienyl)-1,4-phenylenebis(methylene)]-2-(aminomethyl)pyridine;
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-onyl)-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,4,7-triazacyclotetradecanyl)-1,4-phenylenebis(methylene)]-2-(aminomethyl)pyridine;
N-[4-(11-thia-1,4,7-triazacyclotetradecanyl)-1,4-phenylenebis(methylene)]-2-(aminomethyl)pyridine;
N-[4-(11-sulfoxo-1,4,7-triazacyclotetradecanyl)-1,4-phenylenebis(methylene)]-2-(aminomethyl)pyridine;
N-[4-(11-sulfono-1,4,7-triazacyclotetradecanyl)-1,4-phenylenebis(methylene)]-2-(aminomethyl)pyridine; and
N-[4-(3-carboxo-1,4,7-triazacyclotetradecanyl)-1,4-phenylenebis(methylene)]-2-(aminomethyl)pyridine;
or a pharmaceutically acceptable salt thereof.

18. The method of claim 1, further comprising administering in combination a chemotherapeutic agent selected from the group consisting of doxorubicin, epirubicin, cyclophosphamide, methotrexate, 5-fluorouracil, paclitaxel, docetaxel, capecitabine, vinorelbine, gemcitabine and a combination thereof.

19. The method of claim 18, wherein the chemotherapeutic agent is doxorubicin.

20-29. (canceled)

30. The method of claim 18, wherein the compound of formula (1) is selected from 1,1′-[1,4-phenylene-bis-(methylene)-bis-1,4,8,11-tetraazacyclotetradecane and N-[1,4,8,11-tetraazacyclotetradecanyl-(1,4-phenylene-bis-(methylene)]-2-aminoethyl-2-pyridine.

31. The method of claim 1, wherein the breast cancer is selected from the group consisting of lobular carcinoma, ductal carcinoma, inflammatory breast cancer, medullary carcinoma, mucinous (colloid) carcinoma, Paget's disease, tubular carcinoma, phylloides tumor, metaplastic carcinoma, sarcoma, micropapillary carcinoma, adenoid cystic carcinoma, onset breast cancer, relapse breast cancer and refractory breast cancer.

32. The method of claim 1, wherein the CXCR4 antagonist is administered in combination with granulocyte-colony stimulating factor (G-CSF).

33. A pharmaceutical composition for treating breast cancer in unit dosage form, said composition comprising a pharmaceutically effective amount of a CXCR4 antagonist or a pharmaceutically acceptable salt or prodrug thereof and a pharmaceutically acceptable excipient.

34. The pharmaceutical composition of claim 33, wherein the CXCR4 antagonist is a compound of the formula
Z-linker-Z′ (1)
or a pharmaceutically acceptable salt or prodrug thereof,
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
Z′ may be embodied in a form as defined by Z above, or alternatively may be of the formula
—N(R)—(CR₂)ₙ—X
wherein each R is independently H or straight, branched or cyclic alkyl (1-6C), n is 1 or 2, and X is an aromatic ring, including heteroaromatic rings, or is a mercaptan,
or Z′ may be of the formula
—Ar(Y)ⱼ
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

35. The pharmaceutical composition of claim 34, wherein the compound of formula (1) is selected from the group consisting of:
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
methylene (or polymethylene) di 1-N-1,4,8,11-tetraazacyclotetradecane;
11,11′(1,2-ethanediyl)bis-1,4,8,11-tetraazacyclotetradecane;
11,11′(1,2-propanediyl)bis-1,4,8,11-tetraazacyclotetradecane;
11,11′-(1,2-butanediyl)bis-1,4,8,11-tetraazacyclotetradecane;
11,11′(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylene-bis(methylene)]-bis-1,4,8,11-tetraazacyclotetradecane;
1,1′-[1,4-phenylene-bis(methylene)]-bis-1,4,8,11-tetraazacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenyleneis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane; 1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.18,12]tetracosa-1(23),8,10,12(24),19,21-hexaene;
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine;
N-[1,4,8,11-tetraazacyclotetradecanyl-1,4-phenylenebis(methylene)]-4-(amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine;
N-[1,4,8,11-tetraazacyclotetradecanyl-1,4-phenylenebis(methylene)]-2-(amino-ethyl)pyridine;
N-[1,4,8,11-tetraazacyclotetradecanyl-1,4-phenylenebis(methylene)]-2-(amino-methyl)thiophene;
N-[1,4,8,11-tetraazacyclotetradecanyl-1,4-phenylenebis(methylene)]-2-(amino-ethyl)mercaptan;
N-[1,4,8,11-tetraazacyclotetradecanyl-1,4-phenylenebis(methylene)]-2-amino-benzylamine;
N-[1,4,8,11-tetraazacyclotetradecanyl-1,4-phenylenebis(methylene)]-4-amino-benzylamine;
N-[1,4,8,11-tetraazacyclotetradecanyl-1,4-phenylenebis(methylene)]-4-(amino-ethyl)imidazole;
N-[1,4,8,11-tetraazacyclotetradecanyl-1,4-phenylenebis(methylene)]-benzylamine;
N-[1,4,8,11-tetraazacyclotetradecanyl-1,4-phenylenebis(methylene)]-purine;
N-[1,4,8,11-tetraazacyclotetradecanyl-1,4-phenylenebis(methylene)]-4-phenylpiperazine;
1-[2,6-dimethoxypyrid-4-yl(methylene)]-1,4,8,11-tetraazacyclotetradecane;
1-[2-chloropyrid-4-yl(methylene)]-1,4,8,11-tetraazacyclotetradecane;
1-[2,6-dimethylpyrid-4-ylmethylene)]-1,4,8,11-tetraazacyclotetradecane;
1-[2-methylpyrid-4-yl(methylene)]-1,4,8,11-tetraazacyclotetradecane;
1-[2,6-dichloropyrid-4-ylmethylene)]-1,4,8,11-tetraazacyclotetradecane;
1-[2-chloropyrid-5-yl(methylene)]-1,4,8,11-tetraazacyclotetradecane;
7-[4-methylphenyl(methylene)]-4,7,10,17-tetraazabicyclo[13.3.1]heptadeca-1(17),13,15-triene;
N-[4-(1,4,7-triazacyclotetra-decanyl)-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17),13,15-trienyl)-1,4-phenylenebis(methylene)]-2-(aminomethyl)pyridine;
N-[7-(4,7,10-triazabicyclo[13.3.1]heptadeca-1(17),13,15-trienyl)-1,4-phenylenebis(methylene)]-2-(aminomethyl)pyridine;
N-[4-[4,7,10-triazabicyclo[13.3.1]heptadeca-1(17),13,15-trienyl]-1,4-phenylenebis(methylene)]-2-(aminomethyl)pyridine;
N-[4-[4,7,10,17-tetraazabicyclo[13.3.1]heptadeca-1(17),13,15-trienyl]-1,4-phenylenebis(methylene)]-2-(aminomethyl)pyridine;
N-[3-(3,6,17-triazabicyclo[13.3.1]heptadeca-1(17),13,15-trienyl)-1,4-phenylenebis(methylene)]-2-(aminomethyl)pyridine;
N-[3-(3,6,17-triazabicyclo[13.3.1]heptadeca-1(17),13,15-trienyl)-1,3-phenylenebis(methylene)]-2-(aminomethyl)pyridine;
N-[4-(4,7,17-triazabicyclo[13.3.1]heptadeca-1(17),13,15-trienyl)-1,4-phenylenebis(methylene)]-2-(aminomethyl)pyridine;
N-[7-(4,7,17-triazabicyclo[13.3.1]heptadeca-1(17),13,15-trienyl)-1,4-phenylenebis(methylene)]-2-(aminomethyl)pyridine;
N-[6-(3,6,9-triazabicyclo[11.3.1]pentadeca-1(15),11,13-trienyl)-1,3-phenylenebis(methylene)]-2-(aminomethyl)pyridine;
N-[4-(1,7-diazacyclotetradecanyl)-1,4-phenylenebis(methylene)]-2-(aminomethyl)pyridine;
N-[7-(4,10-diazabicyclo[13.3.1]heptadeca-1(17),13,15-trienyl)-1,4-phenylenebis(methylene)]-2-(aminomethyl)pyridine;
N-[7-(4,10,17-triazabicyclo[13.3.1]heptadeca-1(17),13,15-trienyl)-1,4-phenylenebis(methylene)]-2-(aminomethyl)pyridine;
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N14-(1,4,7-triazacyclotetradecan-2-onyl)-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,4,7-triazacyclotetradecanyl)-1,4-phenylenebis(methylene)]-2-(aminomethyl)pyridine;
N-[4-(11-thia-1,4,7-triazacyclotetradecanyl)-1,4-phenylenebis(methylene)]-2-(aminomethyl)pyridine;
N-[4-(11-sulfoxo-1,4,7-triazacyclotetradecanyl)-1,4-phenylenebis(methylene)]-2-(aminomethyl)pyridine;
N-[4-(11-sulfono-1,4,7-triazacyclotetradecanyl)-1,4-phenylenebis(methylene)]-2-(aminomethyl)pyridine; and
N-[4-(3-carboxo-1,4,7-triazacyclotetradecanyl)-1,4-phenylenebis(methylene)]-2-(aminomethyl)pyridine;
or a pharmaceutically acceptable salt thereof.

36. The pharmaceutical composition of claim 34, wherein the compound of formula (1) is selected from 1,1′-[1,4-phenylene-bis-(methylene)-bis-1,4,8,11-tetraazacyclotetradecane or a pharmaceutically acceptable salt thereof and N-[1,4,8,11-tetraazacyclotetradecanyl-(1,4-phenylene-bis-(methylene)]-2-aminoethyl-2-pyridine or a pharmaceutically acceptable salt thereof.

37. The pharmaceutical composition of claim 33, further comprising a chemotherapeutic agent.

38. The pharmaceutical composition of claim 37, wherein the chemotherapeutic agent is selected from the group consisting of doxorubicin, epirubicin, cyclophosphamide, methotrexate, 5-fluorouracil, paclitaxel, docetaxel, capecitabine, vinorelbine, gemcitabine and a combination thereof.

39. A pharmaceutical composition according claim 34, wherein the compound of formula (1) is 1,1′-[1,4-phenylene-bis-(methylene)-bis-1,4,8,11-tetraazacyclotetradecane.
